(19) United States
(12) Patent Application Publication
(10) Pub. No.: US 2019/0029090 A1
(43) Pub. Date: Jan. 24, 2019

(54) LIGHT IRRADIATION SYSTEM, CONTROL APPARATUS, LIGHT IRRADIATION CONTROL METHOD, AND OPERATING MICROSCOPE APPARATUS

(71) Applicants: National Institute of Advanced Industrial Science and Technology, Tokyo (JP); Nikon Corporation, Tokyo (JP)

(72) Inventors: Yuzuru Ikehara, Tsukuba-shi (JP); Susumu Makinouchi, Yokohama-shi (JP); Shunji Watanabe, Tokyo (JP); Tetsuro Ishikawa, Osaka (JP)

(73) Assignees: National Institute of Advanced Industrial Science and Technology, Tokyo (JP); Nikon Corporation, Tokyo (JP)

(21) Appl. No.: 16/138,547

(22) Filed: Sep. 21, 2018

Related U.S. Application Data
(63) Continuation of application No. PCT/JP2017/010846, filed on Mar. 17, 2017.

(30) Foreign Application Priority Data
Mar. 22, 2016 (JP) 2016-057444

(51) Int. Cl. H05B 33/08; A61B 90/30; A61B 5/00; G02B 21/00; G02F 1/137; G02F 1/133; G02B 21/16; H05B 37/02

(52) U.S. Cl. CPC H05B 33/0845; A61B 90/30; A61B 5/0071; G02B 21/0012; A61B 2090/304; G02F 1/13306; G02B 21/16; H05B 37/0272; A61B 2090/309; G02F 1/137

(57) ABSTRACT

In an imaging using fluorescence, for example, it is difficult to perform a surgery while a tumor part is directly confirmed in a bright operating room. A light irradiation system according to an embodiment performs an open/close control of a first eyesight of a first eyesight restrictor and a second eyesight of a second eyesight restrictor at every predetermined time interval. The light irradiation system controls a light-on of a light such that opening and closing timings of the first eyesight match a timing of a light amount control of a surgery lamp and opening and closing timings of the second eyesight match a timing of a light amount control of a fluorescence excitation light source (see FIG. 1).

### CROSS REFERENCE TO RELATED APPLICATIONS

This is a Continuation of PCT International Application PCT/JP2017/010846 filed on Mar. 17, 2017, which in turns claims benefit of Japanese patent application 2016-057444 filed in Japan on Mar. 22, 2016. The entire contents of each of the above documents are hereby incorporated by reference into the present application.

### TECHNICAL FIELD

The present invention relates to a light irradiation system, a control apparatus, a light irradiation control method, and an operating microscope apparatus.

### BACKGROUND ART

Recently, the use of an imaging utilizing fluorescence for identification of a tumor during surgery has been often attempted. For example, Non Patent Literature 1 discloses that after 5-ALA, a fluorescer, is administrated to an affected part of a mouse affected with cancer, fluorescence is recognized from this affected part through an irradiation of the affected part with excitation light. The use of this fluorescence action probably allows a person such as a doctor to perform a surgery while seeing the fluorescence from a tumor part of a patient.

### CITATION LIST

### Patent Literature

Patent Literature 1: JP 2012-98371 A

### Non Patent Literature

Non Patent Literature 1: “Fluorescence Diagnosis of Lymph Node Metastasis of Gastrointestinal Cancers by Using 5-Aminolevulinic Acid (5-ALA)” by Yoshinori Harada, Journal of Kyoto Prefectural University of Medicine, 122(4), 181 to 188, 2013

### SUMMARY OF INVENTION

However, the above-described fluorescence is generally considerably faint compared with a surgery lamp illumination. Therefore, to apply the above-described fluorescence action to a surgery of a patient, the surgery lamp illumination needs to be turned off to set the periphery dark and the surgery needs to be performed with the surgery lamp illumination turned off for observation of the fluorescence. In such case, while the fluorescent part can be confirmed during the surgery, other anatomical structures cannot be confirmed. Accordingly, for example, a doctor cannot perform the surgery while directly confirming a tumor part (the fluorescent part) and the other anatomical structures in an operating room. The doctor or a similar person who performs the surgery desires to ensure directly confirming the tumor part (the fluorescent part) and the other anatomical structures in the bright operating room.

According to this embodiment, there is provided a light irradiation system that includes a light source, an eyesight restricting apparatus, and a control apparatus. The light source is configured to emit a first light and a second light with which a biological tissue is irradiated. The eyesight restricting apparatus includes a first eyesight restrictor and a second eyesight restrictor configured to control eyesights of a wearing target. The control apparatus is configured to control a behavior of the light source and a behavior of the eyesight restricting apparatus. The control apparatus is configured to perform an open/close control on a first eyesight of the first eyesight restrictor and a second eyesight of the second eyesight restrictor at every predetermined time interval. The control apparatus is configured to control a light irradiation behavior by the light source such that: opening and closing timings of any one of the first eyesight and the second eyesight match a timing of a light amount control of the irradiated first light; and opening and closing timings of an eyesight other than the eyesight matched with the timing of the light amount control of the first light match a timing of a light amount control of the irradiated second light.

According to another embodiment, there is provided a control apparatus that controls a behavior of a light source and a behavior of an eyesight restricting apparatus. The light source irradiates a biological tissue with a first light and a second light. The eyesight restricting apparatus includes a first eyesight restrictor and a second eyesight restrictor to control eyesights of a wearing target. The control apparatus includes a memory and a controller. The memory stores a program to control the behavior of the light source and the behavior of the eyesight restricting apparatus. The controller is configured to read the program from the memory to execute the program. The controller is configured to perform an open/close control on a first eyesight of the first eyesight restrictor and a second eyesight of the second eyesight restrictor at every predetermined time interval. The controller is configured to control a light irradiation behavior by the light source such that: opening and closing timings of any one of the first eyesight and the second eyesight match a timing of a light amount control of the irradiated first light; and opening and closing timings of an eyesight other than the eyesight matched with the timing of the light amount control of the first light match a timing of a light amount control of the irradiated second light.

According to another embodiment, there is provided a light irradiation system that includes a light source, an eyesight restricting apparatus, and a control apparatus. The light source is configured to irradiate a biological tissue with a first light and a second light. The eyesight restricting apparatus includes a first eyesight restrictor and a second eyesight restrictor configured to control eyesights of a wearing target. The control apparatus is configured to control a behavior of the light source. The eyesight restricting apparatus is configured to perform an open/close control on a first eyesight of the first eyesight restrictor and a second eyesight of the second eyesight restrictor at every predetermined time interval. The control apparatus is configured to communicate with the eyesight restricting apparatus to acquire information on opening and closing timings of the first eyesight and the second eyesight. The control apparatus is configured to control a light irradiation behavior by the light source such that: opening and closing timings of any one of the first eyesight and the second eyesight match a timing of a light amount control of the irradiated first light; and opening and closing timings of an eyesight other than the eyesight matched with the timing of the light amount control of the first light match a timing of a light amount control of the irradiated second light.

According to another embodiment, there is provided a control apparatus that controls a behavior of a light source. The light source irradiates a biological tissue with a first light and a second light. The control apparatus includes a memory and a controller. The memory stores a program to control the behavior of the light source. The controller is configured to read the program from the memory to execute the program. The controller is configured to acquire information on a timing of an open and close control from an eyesight restricting apparatus. The eyesight restricting apparatus includes a first eyesight restrictor and a second eyesight restrictor to control eyesights of a wearing target. The open/close control is performed on a first eyesight of the first eyesight restrictor and a second eyesight of the second eyesight restrictor at every predetermined time interval. The controller is configured to control a light irradiation behavior by the light source such that: opening and closing timings of any one of the first eyesight and the second eyesight match a timing of a light amount control of the irradiated first light; and opening and closing timings of an eyesight other than the eyesight matched with the timing of the light amount control of the first light match a timing of a light amount control of the irradiated second light.

According to another embodiment, there is provided a light irradiation control method that controls a behavior of a light source and a behavior of an eyesight restricting apparatus by a control apparatus. The light source emits a first light and a second light. The eyesight restricting apparatus includes a first eyesight restrictor and a second eyesight restrictor to control eyesights of a wearing target. The light irradiation control method includes: performing an open/close control on a first eyesight of the first eyesight restrictor and a second eyesight of the second eyesight restrictor at every predetermined time interval by the control apparatus; and controlling a light irradiation behavior by the light source by the control apparatus such that: opening and closing timings of any one of the first eyesight and the second eyesight match a timing of a light amount control of the irradiated first light; and opening and closing timings of an eyesight other than the eyesight matched with the timing of the light amount control of the first light match a timing of a light amount control of the irradiated second light.

According to another embodiment, there is provided a light irradiation control method that controls a behavior of a light source emitting a first light and a second light by a control apparatus. The light irradiation control method includes: communicating with an eyesight restricting apparatus to acquire information on a timing of an open/close control from the eyesight restricting apparatus by the control apparatus, the eyesight restricting apparatus including a first eyesight restrictor and a second eyesight restrictor to control eyesights of a wearing target, the open/close control being performed on a first eyesight of the first eyesight restrictor and a second eyesight of second eyesight restrictor at every predetermined time interval; and controlling a light irradiation behavior by the light source by the control apparatus such that: opening and closing timings of any one of the first eyesight and the second eyesight match a timing of a light amount control of the irradiated first light; and opening and closing timings of an eyesight other than the eyesight matched with the timing of the light amount control of the first light match a timing of a light amount control of the irradiated second light.

According to another embodiment, there is provided an operating microscope apparatus that includes a light introduction mechanism, an eyepiece, a usual light path, a light divider, a first light path and a second light path, an eyesight restrictor, and a controller. The light introduction mechanism is configured to introduce a first light and a second light emitted from a light source to a biological tissue. The usual light path is configured to guide a light from the biological tissue to the eyepiece. The light divider is disposed on the usual light path. The light divider is configured to divide the light from the biological tissue into two lights. The first light path and the second light path are disposed between the light divider and the eyepiece. One light among the two lights passes through the first light path. Another light among the two lights passes through the second light path. The eyesight restrictor includes a first eyesight restrictor disposed at the first light path and a second eyesight restrictor disposed at the second light path. The controller is configured to control a behavior of the light source and a behavior of the eyesight restrictor. The controller is configured to perform an open/close control on a first eyesight of the first eyesight restrictor and a second eyesight of the second eyesight restrictor at every predetermined time interval. The controller is configured to match opening and closing timings of any one of the first eyesight and the second eyesight with a timing of a light amount control of the introduced first light. The controller is configured to match opening and closing timings of an eyesight other than the eyesight matched with the timing of the light amount control of the first light with a timing of a light amount control of the introduced second light.

According to yet another embodiment, there is provided an illumination control system that includes a control apparatus and an eyesight restricting apparatus. The control apparatus is configured to control a behavior of a light source. The eyesight restricting apparatus is configured to control eyesights where two lights at different wavelengths transmit. The eyesight restricting apparatus is configured to adjust light amounts of the two lights transmitting the eyesights.

### BRIEF DESCRIPTION OF DRAWINGS

FIG. 1 is a drawing illustrating a schematic configuration of a surgery assistance system according to this embodiment.

FIG. 2 includes drawings describing an outline of a light irradiation timing control according to first and second embodiments.

FIG. 3 is a timing chart illustrating switching timings to open/close liquid crystal shutters on liquid crystal glasses 40, light-on and off timings of a surgery lamp 20, and light-on and off timings of a fluorescence excitation light source 30 according to the first and second embodiments.

FIG. 4 is a flowchart describing process contents (contents of an illumination control program 1061) of a control apparatus 10 according to the first embodiment.

FIG. 5 is a flowchart describing process contents (contents of the illumination control program 1061) of the control apparatus 10 according to the second embodiment.

FIG. 6 is a schematic diagram illustrating an example of an appearance of an operating microscope system 60 according to a third embodiment.

FIG. 7 is a schematic diagram illustrating an example of an appearance of an operating microscope 70 according to the third embodiment.

FIG. 8 is a drawing illustrating an example of an internal configuration of the operating microscope 70 according to the third embodiment.

FIG. 9 is a drawing illustrating a configuration example when optical systems of the operating microscope 70 according to the third embodiment are disassembled.

FIG. 10 includes drawings describing an outline of a light irradiation timing control according to fourth and fifth embodiments.

FIG. 11 is a timing chart illustrating switching timings to open/close liquid crystal shutters on the liquid crystal glasses 40, light-on and off timings of the surgery lamp 20, and light-on and off timings of the fluorescence excitation light source 30 according to the fourth and the fifth embodiments.

FIG. 12 is a flowchart describing process contents (contents of the illumination control program 1061) of the control apparatus 10 according to the fourth embodiment.

FIG. 13 is a flowchart describing process contents (contents of the illumination control program 1061) of the control apparatus 10 according to the fifth embodiment.

### DESCRIPTION OF EMBODIMENTS

The following describes embodiments with reference to the accompanying drawings. The accompanying drawings represent functionally identical elements by identical reference numerals in some cases. Although the accompanying drawings illustrate the embodiments and examples of implementation according to a principle of the present invention, these drawings are for understanding of the present invention and never used for limited interpretation of the present invention. The descriptions of this description are merely typical examples and therefore do not limit the claims and application examples of the present invention by any means.

While the embodiments give the description in detail enough for a person skilled in the art to carry out this invention, it is necessary to understand that other implementations and configurations are possible and that changes in configurations and structures and substitutions of various components can be made without departing from the scope and spirit of the technical idea of this invention. Therefore, the following description should not be interpreted to be limited.

Further, as described later, the embodiments may be implemented by software running on a general-purpose computer, by dedicated hardware, or by a combination of software and hardware.

### (1) First Embodiment

<Configuration of Surgery Assistance System>

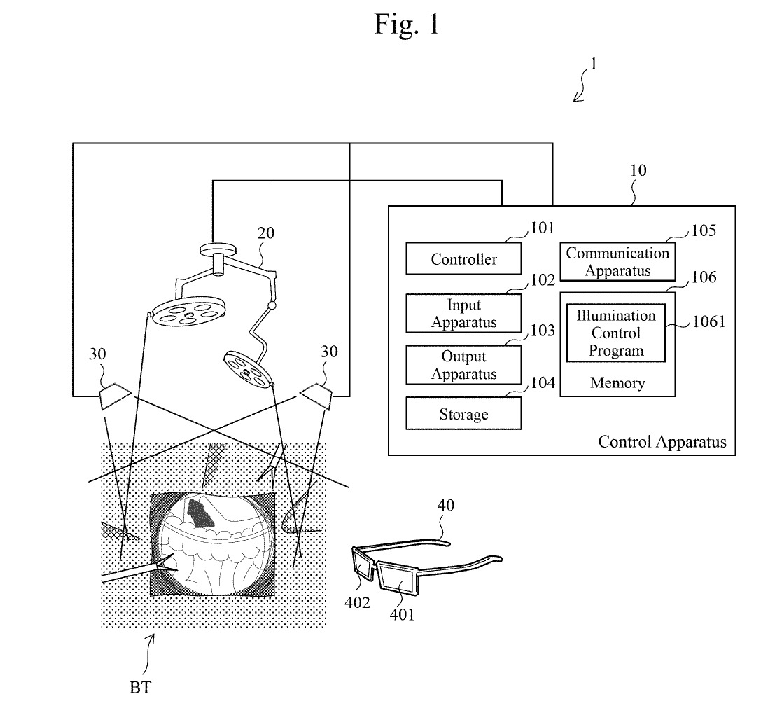

FIG. 1 is a drawing illustrating the configuration of the surgery assistance system (can also be referred to as a medical assistance system or a light irradiation system) according to this embodiment. While this embodiment defines this system as “surgery” application for convenience of description, this system is not limited to for “surgery” and is widely applicable as, for example, “medical treatment” application.

A surgery assistance system 1 is, for example, usable for an abdominal operation for a surgical operation. The surgery assistance system 1 according to the embodiment is, for example, an illumination control system for surgery assistance. The surgery assistance system 1 is to allow a doctor, an operator, or a similar person (can also be simply referred to as “user”) to confirm fluorescence from an affected part, which is obtained through continuous or intermittent irradiations to tissue (an observation target, an irradiated body) BT (for example, an organ: FIG. 1 illustrates an organ as an example of the tissue BT) of an organism (for example, an animal) to which, for example, a fluorescer (as one example, 5-aminolevulinic acid (5-ALA)) reacting to excitation light has been administered (for example, local administration by oral administration and injection) with excitation light under a considerably bright surgery lamp.

As one example, the surgery assistance system 1 includes a control apparatus 10 that controls the entire system, a surgery lamp 20, a fluorescence excitation light source 30, and liquid crystal glasses 40.

The control apparatus 10 includes a controller, an input apparatus 102, an output apparatus 103, a storage 104, a communication apparatus 105, and a memory 106. The controller, which includes a general computer, for example, a processor (such as a CPU and an MPU), reads and performs various kinds of programs from the memory 106. The input apparatus 102, which includes, for example, a changeover switch, a computer mouse, a keyboard, a touch-panel pointer (operated with a stylus pen and a finger), and a microphone, is used to input configuration information, instructions, and data. The output apparatus 103, which includes, for example, a display apparatus, a printer, and a speaker, is used to output data and information. The storage 104 is used to store, for example, process results by the controller and setting contents. The communication apparatus 105 communicates with, for example, an external apparatus and a network. The memory 106 preliminarily stores various kinds of programs and data. The memory 106 holds an illumination control program 1061 to control behaviors of, for example, the surgery lamp 20, the fluorescence excitation light source 30, and the liquid crystal glasses 40.

While in this embodiment, the surgery lamp 20 is a white LED illumination with an illuminance of, for example, 160000 lux, brightness sufficient for surgery, the illumination only needs to have an illuminance of at least, for example, 500 to 1000 lux. The surgery lamp 20 is coupled to the control apparatus 10 such that the control apparatus 10 controls light-on and off timings (a light-on time and a light-off time). For example, the surgery lamp 20, which includes a driver and a controller (not illustrated), turns ON and OFF power of an LED in accordance with the light-on and off timings instructed by the control apparatus 10 causes the surgery lamp 20 to behave so as to repeat light-on and light-off.

The fluorescence excitation light source 30 is a light source that emits a light having a wavelength within a predetermined bandwidth. For example, with the use of the above-described 5-ALA as the fluorescer, as the fluorescence excitation light source 30, a light source with a narrow bandwidth (For example, a width from several nm to several tens of nm may be provided to the wavelength of the emitted light. Alternatively, for example, a wavelength of the excitation light may be changed during the light irradiation.) emitting light (ultraviolet light) with a wavelength of 385 nm to 425 nm is usable. Similar to the surgery lamp 20, the fluorescence excitation light source 30 is coupled to the control apparatus 10 such that the control apparatus 10 controls the light-on and off timings (a light-on time and a light-off time). For example, the fluorescence excitation light source 30, which includes a driver and a controller (not illustrated), turns ON and OFF power of the light source in accordance with the light-on and off timings instructed by the control apparatus 10 causes the fluorescence excitation light source 30 to behave so as to repeat light-on and light-off. As described later, the light-on and off timings of the surgery lamp 20 and the light-on and off timings of the fluorescence excitation light source 30 are controlled such that the light-on and off timings occur in alternation at every predetermined period (for example, 1/60 seconds) or partially overlap with one another.

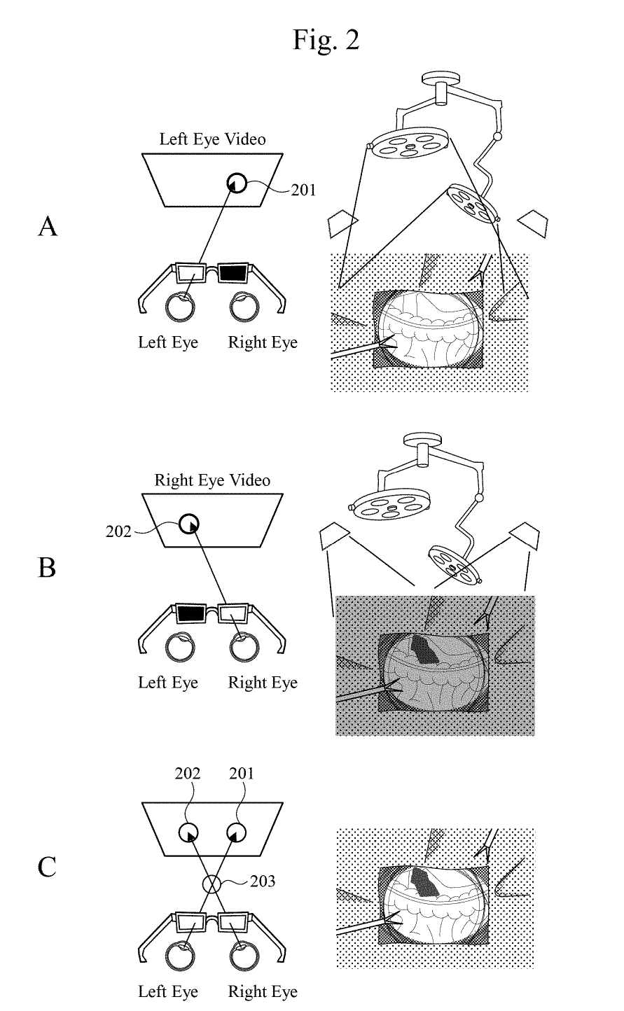

A wearing target (for example, a wearing person and a wearing object) such as the doctor and the operator wears the liquid crystal glasses 40 with which an eyesight of this wearing target (for example, a first eyesight and a second eyesight) can be restricted during, for example, a surgery (a medical practice). The liquid crystal glasses 40 may have a shape of glasses (see FIG. 1, FIG. 2, and a similar drawing) and may have a shape like ski goggles (not illustrated). The liquid crystal glasses 40 with, for example, the shape of glasses include a left eye eyesight restrictor (a first eyesight restrictor) 402 and a right eye eyesight restrictor (a second eyesight restrictor) 401. The left eye eyesight restrictor 402 controls and/or restricts one eyesight (for example, eyesight of a left eye) of the wearing target (for example, the wearing person). The right eye eyesight restrictor 401 controls and/or restricts the other eyesight (for example, eyesight of a right eye) of the wearing target (for example, the wearing person). For example, the liquid crystal glasses 40 with the shape like ski goggles include a left eye eyesight restricting region (a first eyesight restrictor) (not illustrated) and a right eye eyesight restricting region (a second eyesight restrictor) (not illustrated). The left eye eyesight restricting region controls and/or restricts one eyesight (for example, eyesight of a region corresponding to the left eye of the wearing person) of the wearing target. The right eye eyesight restricting region controls and/or restricts the other eyesight (for example, eyesight of a region corresponding to the right eye of the wearing person) of the wearing target.

For example, when the wearing target such as the doctor wears the liquid crystal glasses 40, the liquid crystal glasses 40 are configured so as to ensure wireless communications with the control apparatus 10 via the communication apparatus 105 such that the control apparatus 10 controls a timing at which the eyesight of the left eye and the eyesight of the right eye of the wearing person are restricted in units of predetermined periods (for example, 1/60 seconds). For example, the liquid crystal glasses 40 are configured such that a liquid crystal corresponding to the left eye and a liquid crystal corresponding to the right eye are switched in alternation between a light shielding state and a light transmission state in units of predetermined periods. For example, while the liquid crystal glasses 40 in a first state shade (for example, the liquid crystal glasses 40 adjust a light amount at the first eyesight) the one eyesight (for example, the eyesight of the left eye) of the wearing person dark by light shielding of the first eyesight (for example, the eyesight corresponding to the left eye of the wearing target) of the first eyesight restrictor 402, the liquid crystal glasses 40 set the other eyesight (for example, the eyesight of the right eye) of the wearing person so as to be brightly seeable (for example, the liquid crystal glasses 40 adjust the light amount at the second eyesight so as to be a light amount different from the light amount of the first eyesight) by light transmission of the second eyesight (for example, the eyesight corresponding to the right eye of the wearing target) of the second eyesight restrictor 401. Additionally, after a lapse of a predetermined period, while the liquid crystal glasses 40 in a second state set the one eyesight of the wearing person so as to be brightly seeable by the light transmission of the first eyesight of the first eyesight restrictor 402, the liquid crystal glasses 40 shade the other eyesight of the wearing person dark by the light shielding of the second eyesight of the second eyesight restrictor 401. The liquid crystal glasses 40 according to this embodiment control the light transmission of the liquid crystal region (the first eyesight restrictor) corresponding to the one eyesight (for example, the eyesight of the left eye) of the wearing person and the liquid crystal region (the second eyesight restrictor) corresponding to the other eyesight (for example, the eyesight of the right eye) of the wearing person, thus ensuring switching between the above-described first state and the above-described second state in alternation in units of predetermined periods. While this embodiment uses the liquid crystal glasses 40, as long as the light shielding and the light transmission states (for example, including the light amounts at the respective eyesights) can be switched in alternation between the first eyesight (for example, the eyesight corresponding to the left eye) and the second eyesight (for example, the eyesight corresponding to the right eye), a principle other than the liquid crystal may be used. As an apparatus using a principle other than the liquid crystal, for example, a glasses-type apparatus that switches the light shielding and the light transmission states between the first eyesight and the second eyesight in alternation by, for example, a Digital Micromirror Device (DMD) may be used. In this embodiment, the liquid crystal glasses 40 and the DMD glasses-type apparatus can be collectively referred to as, for example, an eyesight restricting apparatus. In addition to the glasses-type apparatus, for example, a head mounted eyesight restricting apparatus may be used. As one example, this embodiment can be described using the eyesight of the left eye and the eyesight of the right eye of the wearing target.

The control apparatus 10 communicates with the respective surgery lamp 20, fluorescence excitation light source 30, and liquid crystal glasses 40 to instruct the respective drivers (not illustrated) and controllers (not illustrated) on the light-on and off timings and opening and closing timings of liquid crystal shutters (hereinafter simply referred to as “shutters” in some cases) on the liquid crystal glasses 40. For example, when the eyesight of the left eye is in the light shielding state (the left eye shutter closed) and the right eye is in the light transmission state (the right eye shutter opened) in the liquid crystal glasses 40, the control apparatus 10 controls the respective behaviors such that the surgery lamp 20 lights off and the fluorescence excitation light source 30 lights on. Additionally, for example, when the eyesight of the left eye is in the light transmission state (the left eye shutter opened) and the right eye is in the light shielding state (the right eye shutter closed), the control apparatus 10 controls the respective behaviors such that the surgery lamp 20 lights on and the fluorescence excitation light source 30 lights off. While the control apparatus 10, the surgery lamp 20, and the fluorescence excitation light source 30 are coupled together by wires in FIG. 1, for example, the control apparatus 10 may control the surgery lamp 20 and the fluorescence excitation light source 30 through wireless communications. The liquid crystal glasses 40 that have received a control signal regarding the behaviors of the opening and closing timings of the above-described shutters from the control apparatus 10 controls the light transmission and/or the light shielding of the first eyesight of the first eyesight restrictor 402 corresponding to the eyesight of the left eye and the light transmission and/or the light shielding of the second eyesight of the second eyesight restrictor 401 corresponding to the eyesight of the right eye based on the received control signal regarding the opening and closing timings. For example, the liquid crystal glasses 40 include the two liquid crystal shutters (the left eye shutter and the right eye shutter) as the eyesight restrictors (the first eyesight restrictor, the second eyesight restrictor). The opening and closing behaviors of the respective shutters control the light transmission and/or the light shielding of the first eyesight restrictor 402 and the light transmission and/or the light shielding of the second eyesight restrictor 401.

While this embodiment describes the example of the light source unis of the surgery lamp 20 and the fluorescence excitation light source 30 disposed as the respective independent light sources, for example, one light source may emit (for example, the lights need to be controllable so as to be irradiated in alternation or the irradiation timings of the lights need to be controllable so as to partially overlap with one another) an illuminating light for surgery (a light with illuminance of, for example, 500 to 1000 lux or more) and an excitation light (a light with a wavelength of, for example, 385 nm to 425 nm).

<Process Outline>

FIG. 2 includes drawings describing an outline of a light irradiation timing control. Here illustrates an example in which the light irradiation by the surgery lamp 20 and the irradiation of the excitation light by the fluorescence excitation light source 30 are performed in alternation. FIG. 2(A) illustrates a left eye video (an odd number field) 201 seen by the doctor or a similar person who wears the liquid crystal glasses 40 through the left eye shutter (the left eyesight) in the light transmission state for 1/60 seconds during which the surgery lamp 20 lights on. FIG. 2(B) illustrates a right eye video (an even number field) 202 seen through the right eye shutter (the right eyesight) where the doctor or a similar person who wears the liquid crystal glasses 40 is in the light transmission state for 1/60 seconds during which the tissue BT is irradiated with the excitation light from the fluorescence excitation light source 30. FIG. 2(C) illustrates an image 203 combined in a brain of the doctor or a similar person who wears the liquid crystal glasses 40.

As illustrated in FIG. 2, the control apparatus 10 controls (see FIG. 2(A) and FIG. 2(B)) the liquid crystal shutters of the liquid crystal glasses 40 such that the openings and closings of the left eye shutter (the eyesight of the left eye (can also be referred to as the first eyesight)) and the right eye shutter (the eyesight of the right eye (can also be referred to as the second eyesight)) are switched in alternation, for example, in units of 1/60 seconds. As one example, with the left eye shutter opened, the surgery lamp 20 is controlled to light on such that the tissue BT is irradiated with the illuminating light (for example, a white LED light). For example, an image captured by the left eye constitutes an image 201 in the odd number field and an image captured by the right eye constitutes the image 202 in the even number field. For example, the image of the right eye and the image of the left eye are seen to be combined in a pseudo manner, and thus the fluorescence from the tissue BT is seen to be highlighted (see FIG. 2(C)). The image (the combined image) combined in the brain of the doctor or a similar person becomes a frame image (a played image) 203 in units of 1/30.

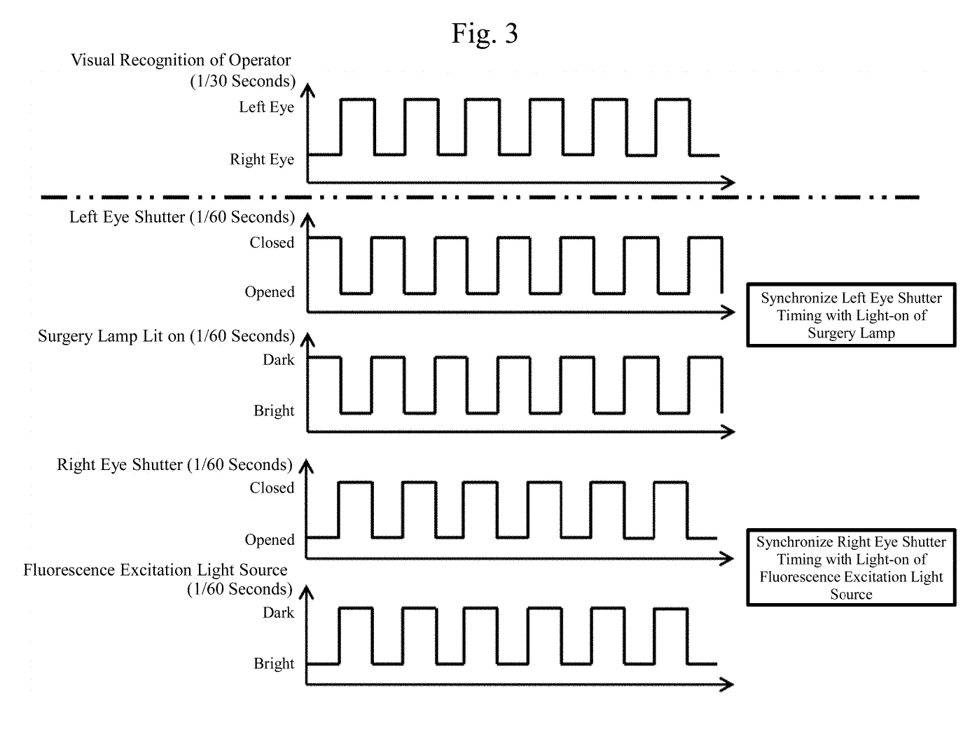

FIG. 3 is a timing chart illustrating the switching timings to open/close the liquid crystal shutters on the liquid crystal glasses 40, the light-on and off timings of the surgery lamp 20, and the light-on and off timings of the fluorescence excitation light source 30. While FIG. 3 illustrates the case where the light irradiation by the surgery lamp 20 and the irradiation of the excitation light by the fluorescence excitation light source 30 are performed in alternation as one example, the timing of the light irradiation by the surgery lamp 20 and the timing of the irradiation of the excitation light by the fluorescence excitation light source 30 may partially overlap with one another.

In FIG. 3, for example, the control apparatus 10 performs the wireless communications with the liquid crystal glasses 40 and controls the opening and closing of the liquid crystal shutters in accordance with this timing chart. As illustrated in FIG. 3, the left eye shutter repeats the opening and closing at every 1/60 seconds. While the right eye shutter repeats the opening and closing at every 1/60 seconds as well, the opening and closing timings are opposite to those of the left eye shutter. For example, while the left eye shutter is open, the surgery lamp 20 is turned ON (bright), and while the left eye shutter is closed, the surgery lamp 20 is turned OFF (dark). While the right eye shutter is open, the fluorescence excitation light source 30 is turned ON (bright), and while the right eye shutter is closed, the fluorescence excitation light source 30 is turned OFF (dark). For example, the control is performed such that the opening and closing timings of the left eye shutter (the first eyesight, the eyesight of the left eye) becomes identical to the ON and OFF timings of the surgery lamp 20 and the opening and closing timings of the right eye shutter (the second eyesight, the eyesight of the right eye) becomes identical to the ON and OFF timings of the fluorescence excitation light source 30. In the case where the surgery lamp 20 lights on (the fluorescence excitation light source 30 lights off) and the left eye shutter is open, which is in the light transmission state, the light (the illuminating light) of the surgery lamp 20 irradiating the tissue BT transmits the left eye shutter. This allows the doctor or a similar person (an operator) who wears the liquid crystal glasses 40 to observe the tissue BT brightly by the left eye. Meanwhile, when the fluorescence excitation light source 30 lights on (the surgery lamp 20 lights off) and the right eye shutter is open, which is the light transmission state, the irradiation of the tissue BT with the excitation light causes the fluorescence generated from the tissue BT to transmit the right eye shutter. This allows the doctor or a similar person (the operator) who wears the liquid crystal glasses 40 to observe a tumor part (an affected part) emitting the fluorescence in dark eyesight by the right eye. By switchingly controlling these states at every constant period (for example, every 1/60 seconds), the image of the right eye and the image of the left eye are seen to be combined in a pseudo manner in the brain of the doctor or a similar person (the operator), and thus the fluorescence from the tumor part (the affected part) of the tissue BT is seen to be highlighted (The fluorescence from the tissue BT is not negated by the surgery lamp 20 but is clearly seen).

The control apparatus 10 may perform the control so as to match the opening and closing timings of the left eye shutter (the first eyesight, the eyesight of the left eye) with the ON and OFF timings of the fluorescence excitation light source 30 and match the opening and closing timings of the right eye shutter (the second eyesight, the eyesight of the right eye) with the ON and OFF timings of the surgery lamp 20 through communications with the surgery lamp 20, the fluorescence excitation light source 30, and the liquid crystal glasses 40.

The roles of the left eye shutter and the right eye shutter may be switched at a predetermined timing. For example, the control apparatus 10 may perform the control so as to match the opening and closing timings of the left eye shutter with the ON and OFF timings of the fluorescence excitation light source 30 and match the opening and closing timings of the right eye shutter with the ON and OFF timings of the surgery lamp 20 for a constant period through communications with the liquid crystal glasses 40 and the surgery lamp 20. The control apparatus 10 may perform the control so as to match the opening and closing timings of the left eye shutter with the ON and OFF timings of the surgery lamp 20 and match the opening and closing timings of the right eye shutter with the ON and OFF timings of the fluorescence excitation light source 30, for example, after a lapse of a constant period. The control apparatus 10 repeats this control to switch the roles. This configuration ensures avoiding a problem of, for example, losing a sense of perspective caused by seeing the tissue BT only by one eye of the wearing target.

<Process Contents by Control Apparatus: Contents of Light Irradiation Control Process by Illumination Control Program>

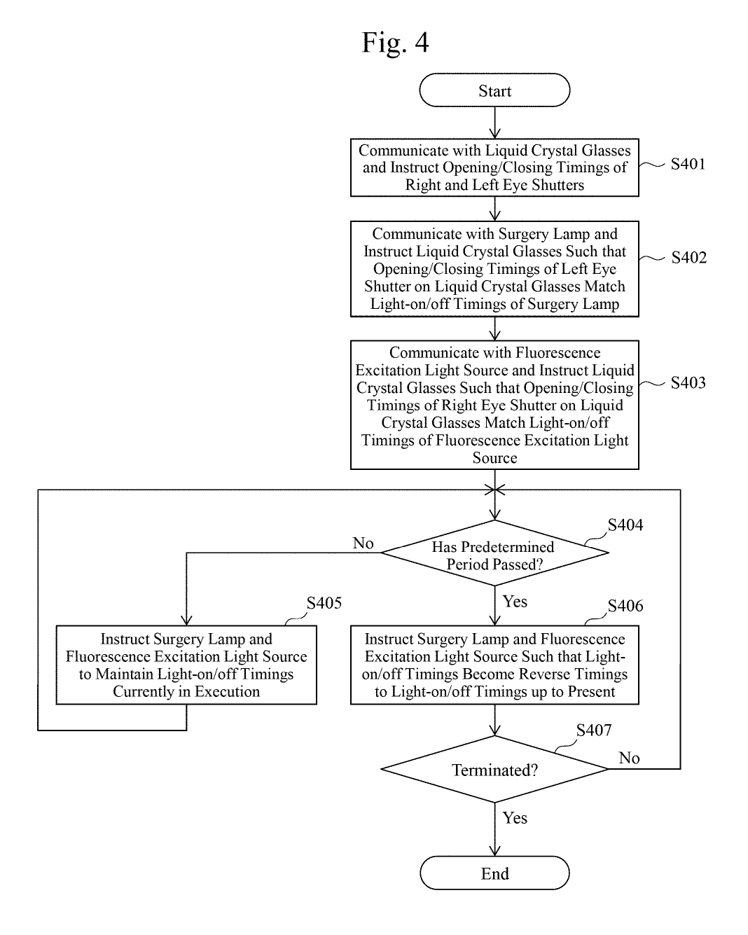

FIG. 4 is a flowchart describing the process contents (the contents of the illumination control program 1061) of the control apparatus 10 according to the first embodiment. A controller 101 in the control apparatus 10 reads the illumination control program 1061 from, for example, a memory (for example, a Read Only Memory (ROM)) into an internal memory (not illustrated) disposed inside the controller 101 to perform the illumination control program 1061. For example, the controller 101 performs the following respective steps in accordance with this illumination control program 1061.

### (i) Step 401

For example, the wearing target, such as the doctor (the operator) who wears the liquid crystal glasses 40 and is going to perform a surgery, turns ON power of the liquid crystal glasses 40, turns ON power of the control apparatus 10, and sets a mode to a surgery mode. Then, the controller 101 in the control apparatus 10 starts communicating with the liquid crystal glasses 40 and instructs the liquid crystal glasses 40 such that the opening and closing timings of the left eye shutter and the right eye shutter on the liquid crystal glasses 40 become the timings illustrated in FIG. 3 by transmission of the control signal.

The liquid crystal glasses 40 sequentially open/close the left eye shutter and the right eye shutter in accordance with the opening and closing timings of the shutters received from the control apparatus 10. The right and left eye shutters on the liquid crystal glasses 40 are opened/closed at, for example, every 1/60 seconds in alternation (see FIG. 3).

### (ii) Step 402

The controller 101 communicates with the surgery lamp 20 and instructs the liquid crystal glasses 40 such that the opening and closing timings of the left eye shutter on the liquid crystal glasses 40 match the light-on and light-off timings of the surgery lamp 20 by transmission of the control signal.

The surgery lamp 20 behaves the driver or the controller (not illustrated) in accordance with the timings of the light-on and the light-off notified from the control apparatus 10 to light on and light off the white LED according to the opening and closing timings of the left eye shutter on the liquid crystal glasses 40. Similar to the left eye shutter on the liquid crystal glasses 40, the timings of the light-on and light-off of the white LED become, for example, every 1/60 seconds.

(iii) Step 403

The controller 101 communicates with the fluorescence excitation light source 30 and instructs the liquid crystal glasses 40 such that the opening and closing timings of the right eye shutter on the liquid crystal glasses 40 match the light-on and light-off timings of the fluorescence excitation light source 30 by transmission of the control signal.

The fluorescence excitation light source 30 behaves the driver or the controller (not illustrated) in accordance with the timings of the light-on and light-off notified from the control apparatus 10 to turn ON and OFF the excitation light irradiation according to the opening and closing timings of the right eye shutter on the liquid crystal glasses 40. Similar to the right eye shutter on the liquid crystal glasses 40, the ON and OFF timings of the excitation light irradiation become, for example, every 1/60 seconds.

### (iv) Step 404

The controller 101 determines whether a predetermined period (for example, may be set to one minute. The setting period is changeable by a user.) has passed from a start of a process of timing synchronization between the surgery lamp 20, the fluorescence excitation light source 30, and the liquid crystal glasses 40 or not. When the predetermined period has passed (Yes at Step 404), the process by the controller 101 transitions to Step 406. When the predetermined period has not passed (No at Step 404), the process by the controller 101 transitions to Step 405.

### (v) Step 405

The controller 101 instructs the surgery lamp 20 and the fluorescence excitation light source 30 to continue the behaviors at the light-on and light-off timings currently in execution.

The surgery lamp 20 and the fluorescence excitation light source 30 receive this instruction to continue the behaviors at the light-on and light-off timings currently in execution from the control apparatus 10 to continue the behaviors in accordance with the instruction.

### (vi) Step 406

The controller 101 instructs the surgery lamp 20 and the fluorescence excitation light source 30 such that the light-on and light-off timings up to the present become the reverse timings to the timings up to the present by transmission of the control signal. Since the opening and closing timings of the shutters on the liquid crystal glasses 40 are identical to those up to the present, the opening and closing timings are not especially instructed.

For example, in the case where the light-on timing and the light-off timing of the surgery lamp 20 has matched the opening and closing timings of the left eye shutter on the liquid crystal glasses 40 and the light-on timing and the light-off timing of the fluorescence excitation light source 30 has matched the opening and closing timings of the right eye shutter on the liquid crystal glasses 40 up to the present, the controller 101 instructs the surgery lamp 20 and the fluorescence excitation light source 30 such that the light-on timing and the light-off timing of the surgery lamp 20 match the opening and closing timings of the right eye shutter and the light-on timing and the light-off timing of the fluorescence excitation light source 30 match the opening and closing timings of the left eye shutter by transmission of the control signal.

The surgery lamp 20 and the fluorescence excitation light source 30 each receive the instructions (the control signals) to change the timings from the control apparatus 10 and change the timings of the light-on and light-off in accordance with the instructions.

(vii) Step 407

The controller 101 determines whether the user (the doctor or a similar person as the operator) has input an instruction (a termination signal) to terminate the process from the input apparatus 102 or not. When the instruction to terminate the process has not been input (No at Step 407), the process transitions to Step 404 to continue the process. When the instruction to terminate the process has been input (Yes at Step 407), the light irradiation control process by this illumination control program 1061 is terminated.

While FIG. 4 represents the sequential execution of the processes of Steps 401 to 403, the processes of these steps may be performed almost simultaneously. Additionally, the processes of Steps 404 and 405 are not essential, and Step 406 may be performed after Step 403. When the process is not terminated, the similar behaviors are continued at the surgery lamp 20, the fluorescence excitation light source 30, and the liquid crystal glasses 40.

In the first embodiment, the control apparatus 10 performs the open/close control on the eyesight of the left eye and the eyesight of the right eye of the wearing target of the liquid crystal glasses (the eyesight restricting apparatus) 40 in alternation at every predetermined time interval and controls these behaviors such that the irradiation of the light (first light) by the light sources (the surgery lamp 20 and the fluorescence excitation light source 30) and the irradiation of the excitation light (second light) occur in alternation. The control apparatus 10 controls the light-on of the lights such that any one of the opening and closing timings of the first eyesight (for example, equivalent to the eyesight of the left eye) of the first eyesight restrictor and the second eyesight (for example, equivalent to the eyesight of the right eye) of the second eyesight restrictor match the ON and OFF timings of the surgery lamp 20; and the opening and closing timings of the eyesight (for example, the second eyesight, the eyesight of the right eye) other than the eyesight (for example, the first eyesight, the eyesight of the left eye) matched with the ON and OFF timings of the surgery lamp 20 match the ON and OFF timings of the fluorescence excitation light source 30. When the eyesight matched with the ON and OFF timings of the surgery lamp 20 is the eyesight of the right eye, the eyesight other than the above-described eyesight becomes the eyesight of the left eye. For example, it can also be expressed that the control apparatus 10 performs the open and close control on the first eyesight and the second eyesight of the liquid crystal glasses (the eyesight restricting apparatus) 40 in alternation at every predetermined time interval and controls these behaviors such that the irradiation of the light (the first light) by the surgery lamp 20 and the irradiation of the excitation light (the second light) by the fluorescence excitation light source 30 are performed in alternation. In this case, the control apparatus 10 controls the light-on of the lights such that the opening and closing timings of the first eyesight match the ON and OFF timings of the surgery lamp 20 and the opening and closing timings of the second eyesight match the ON and OFF timings of the fluorescence excitation light source 30. For example, as the surgery lamp 20, a light source (for example, an LED light source emitting a white light) emitting a visible light is usable, and as the fluorescence excitation light source 30, an excitation light source emitting the excitation light at a predetermined wavelength is usable. In the respective embodiments, the light emission timings of the visible light (the first light) by the surgery lamp and the excitation light (the second light) by the excitation light source may occur at cycles of mutually emitting the lights in alternation, may occur at cycles in phases opposite to one another, or may occur at cycles of partially overlapping with one another. According to this embodiment, the user (the doctor or a similar person) needs not to observe the fluorescence emitted from a biological tissue in the dark periphery, ensuring the surgery while directly confirming a characterizing part (for example, the tumor part) in the bright operating room. Note that the light sources (as described above, the one light source may switchingly irradiate the visible light and the excitation light) according to this embodiment each irradiate the biological tissue to which the fluorescer has been administrated with the excitation light and the visible light. With the light irradiation system according to the embodiment, the irradiation of the excitation light emits the fluorescence from the tumor part of the biological tissue, thus allowing the user to observe the affected part (the characterizing part, the generated fluorescence) in a bright room.

As the excitation light emitted by the fluorescence excitation light source 30, the ultraviolet light with the wavelength of, for example, 385 nm to 425 nm is usable. The ultraviolet light cannot be seen by the eyes, and therefore this ensures preventing the excitation light from irradiating not only the affected part but also the entire biological tissue (for example, an organ) with color and hindering the medical practice.

As described in this embodiment, as the eyesight restricting apparatus, the liquid crystal glasses 40, for example, that can switch the right and left eyesights between the transmission and the light shielding in alternation is usable. Besides, the liquid crystal glasses 40 may have the shape like ski goggles. The use of the glasses-shaped and the ski-goggle-shaped apparatuses facilitates wearing the eyesight restricting apparatus by the doctor and the operator, and the wearing of this apparatus does not hinder the medical practice. The eyesight restricting apparatus (or the eyesight restrictors) according to this embodiment, for example, may be configured to include an optical member such as a wavelength filter (for example, a visible filter, a bandpass filter, and a dichroic mirror), which has a function that can control the eyesights of the user by controlling (or restricting) the transmitting light and selects the wavelength of the transmitting light; and a filter (for example, an ND filter and a polarization filter), which adjusts (for example, dims the transmission light) the light amount of the transmitting light.

The control apparatus 10 performs, for example, the wireless communications with the eyesight restricting apparatus (for example, the liquid crystal glasses 40) to perform the open/close control on the eyesight of the left eye and the eyesight of the right eye. The opening and closing timings of the eyesight of the left eye and the eyesight of the right eye in this eyesight restricting apparatus, for example, may be controlled so as to occur in alternation or may partially overlap with one another. The timings partially overlap with one another mean that, for example, there are time slots in which both of the eyesight of the right eye and the eyesight of a left eye are in the open state and the closed state, or there is a time slot in which both eyesights are in the closed state or the open state. Although not illustrated, the liquid crystal glasses 40 at least include a communication apparatus to communicate with the control apparatus 10, a processor that performs an instruction from the control apparatus 10, and a power supply to drive these apparatuses. Since the control apparatus 10 and the eyesight restricting apparatus (the liquid crystal glasses 40) can communicate with one another by wireless communications, the doctor and the operator who wear the eyesight restricting apparatus can smoothly move, and this eliminates the interference to the medical practice such as the surgery due to wiring and the like.

### (2) Second Embodiment

While the control apparatus 10 instructs the surgery lamp 20, the fluorescence excitation light source 30, and the liquid crystal glasses 40 on the respective behavior timings in the first embodiment, switching ON the liquid crystal glasses 40 according to the second embodiment, for example, autonomously opens/closes the left eye shutter and the right eye shutter at predetermined timings. Through communications with the liquid crystal glasses 40, the control apparatus 10 senses (acquires information on the opening and closing timings) the opening and closing timings of the right and left eye shutters on the liquid crystal glasses 40. The configuration (see FIG. 1) of the surgery assistance system 1 and the timing chart (FIG. 3) of the second embodiment are similar to those of the first embodiment.

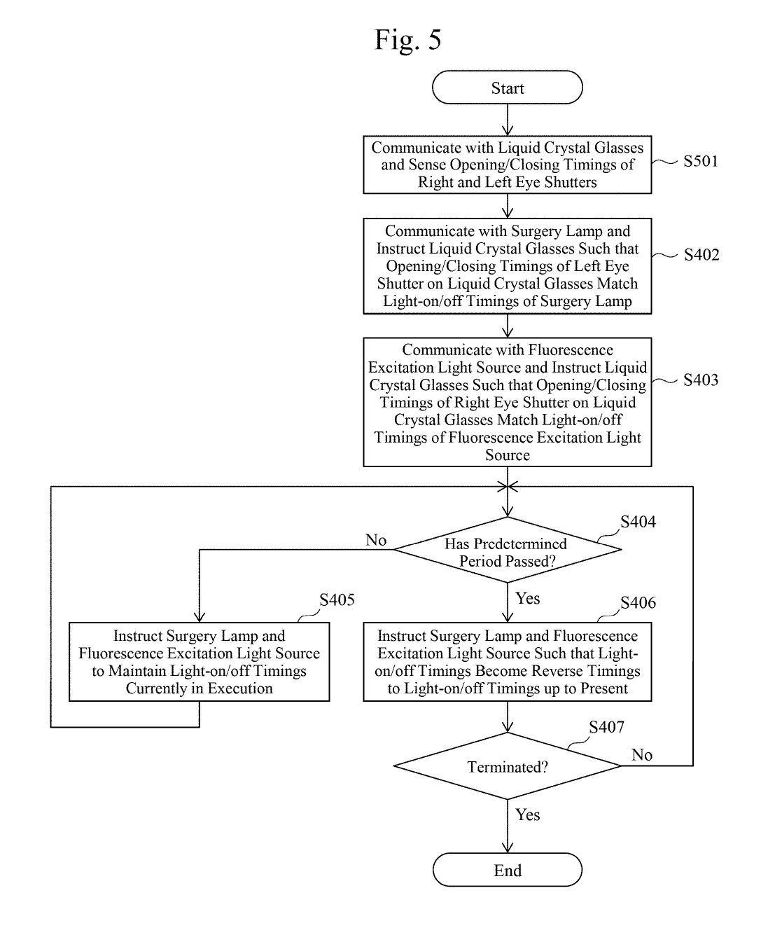

FIG. 5 is a flowchart describing process contents (contents of the illumination control program 1061) of the control apparatus 10 according to the second embodiment. FIG. 5 differs from FIG. 4 only in Step 501, and the other processes of FIG. 5 are similar to those of FIG. 4.

### (i) Step 501

For example, the wearing target, such as the doctor (the operator) who wears the liquid crystal glasses 40 and is going to perform a surgery, turns ON power of the liquid crystal glasses 40, turns ON power of the control apparatus 10, and sets a mode to a surgery mode. Then, the liquid crystal glasses 40 start the opening and closing behaviors of the right and left eye shutters at preliminarily set timings (for example, every 1/60 seconds). The controller 101 in the control apparatus 10 communicates with the liquid crystal glasses 40 and senses the opening and closing timings of the left eye shutter and the right eye shutter on the liquid crystal glasses 40. For example, in the liquid crystal glasses 40, a memory (not illustrated) holds information on the opening and closing timings of the liquid crystal shutters for the right and left eyes and a controller (a processor, not illustrated) acquires the information on the opening and closing timings of the liquid crystal shutters from this memory, thus actually opening and closing the liquid crystal shutters for the right and left eyes. Therefore, the control apparatus 10 communicates with the controller in the liquid crystal glasses 40 using the communication apparatus 105 to acquire the information on the opening and closing timings of the liquid crystal shutters that actually behave. The opening and closing timings of the shutters for the right and left eyes thus acquired are as illustrated in FIG. 3 (for example, the shutters are opened/closed at every 1/60 seconds in alternation).

### (ii) Steps 402 to 407

Since being similar to those of FIG. 4, the process contents of Steps 402 to 407 are omitted. While the processes of Steps 401 to 403 may be performed simultaneously in the first embodiment, the process of Step 501 needs to be performed first to acquire the information on the opening and closing timings of the right and left eye shutters on the liquid crystal glasses 40 in the second embodiment. The light-on and off timings of the surgery lamp 20 and the fluorescence excitation light source 30 are controlled according to the information. Note that the processes of Steps 402 and 403 may be performed simultaneously.

In the second embodiment, the eyesight restricting apparatus (for example, the liquid crystal glasses 40) performs the open/close control on the eyesight of the left eye and the eyesight of the right eye of the person (for example, the doctor and the operator) who wears the eyesight restricting apparatus at every predetermined time interval. While the eyesight restricting apparatus (for example, the liquid crystal glasses 40) may perform the open/close control on the eyesight of the left eye and the eyesight of the right eye in alternation as described above, the open/close control may be performed such that the opening and closing timings of the eyesight of the left eye and the eyesight of the right eye at this eyesight restricting apparatus partially overlap with one another. The case where the timings partially overlap with one another can include, for example, the case where there are time slots in which both of the eyesight of the right eye and the eyesight of the left eye are in the open state and the closed state, or there is a time slot in which both eyesights are in the closed state or the open state. The eyesight of the left eye can also be referred to as the first eyesight and the eyesight of the right eye as the second eyesight, or conversely, the eyesight of the right eye can also be referred to as the first eyesight and the eyesight of the left eye as the second eyesight. For example, while the control apparatus 10 controls this open/close control in the first embodiment, the eyesight restricting apparatus performs this open/close control by itself in the second embodiment. Meanwhile, the control apparatus 10 controls the irradiation timing of the first light and the irradiation timing of the second light by the light sources (the surgery lamp 20 and the fluorescence excitation light source 30). In such second embodiment, the control apparatus 10 communicates (for example, the wireless communications) with the eyesight restricting apparatus (for example, the liquid crystal glasses 40) to acquire the information on the opening and closing timings of the first eyesight and the second eyesight of the eyesight restricting apparatus (the liquid crystal glasses 40). Accordingly, the control apparatus 10 can acquire the information on the opening and closing timings of the respective eyesights of the eyesight restricting apparatus (the liquid crystal glasses 40). The control apparatus 10 controls the light-on of the lights by the light source unis (the surgery lamp 20 and the fluorescence excitation light source 30) such that the first light irradiation occurs in alternation with the second light irradiation, the opening and closing timings of the first eyesight match the ON and OFF timings of the first light irradiation, and the opening and closing timings of the second eyesight match the ON and OFF timings of the second light irradiation. According to the second embodiment, the eyesight restricting apparatus (the liquid crystal glasses 40) needs not to control the behaviors according to the control signal from the control apparatus 10. This allows further simplification of the apparatus internal configuration of the eyesight restricting apparatus (the liquid crystal glasses 40).

### (3) Third Embodiment

The third embodiment relates to an operating microscope to which the principles of the first and second embodiments are applied. To perform the surgery on the patient (for example, a human and an animal) using this operating microscope, for example, a fluorescer (as one example, 5-aminolevulinic acid (5-ALA)) reacting to excitation light is administrated to the patient at a predetermined time before the surgery. For example, a local administration by oral administration and injection is considered as the administration method. Thus, this operating microscope is used after administrating, for example, the predetermined fluorescer.

<Appearance Configuration>

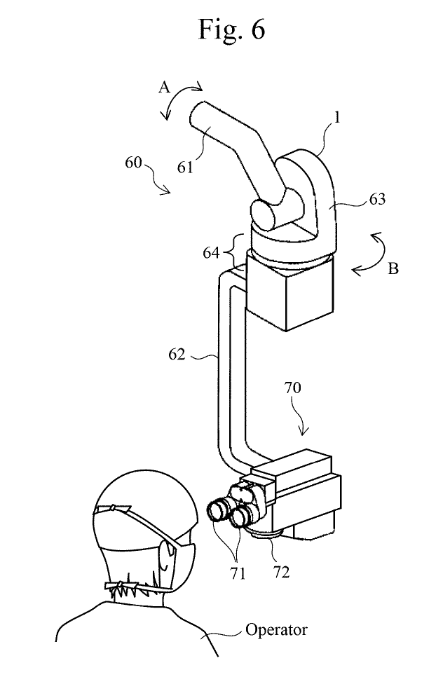

FIG. 6 is a schematic diagram illustrating an example of an appearance of an operating microscope system 60 according to the third embodiment. The operating microscope system 60 includes an operating microscope 70, a support arm 62 that supports this operating microscope 70, a distal end member 63, a link mechanism 64 that couples the support arm 62 and the distal end member 63 together, and a coupling arm 61 to couple the distal end member 63 and a stand apparatus (not illustrated) together. The coupling arm 61 couples the stand apparatus (not illustrated) and the distal end member 63 together so as to be rotatable, for example, in an arrow A direction. For example, the height may be adjusted with the stand apparatus (not illustrated). The link mechanism 64 is a mechanism, for example, to configure the support arm 62 and the distal end member 63 to be rotatable in an arrow B direction.

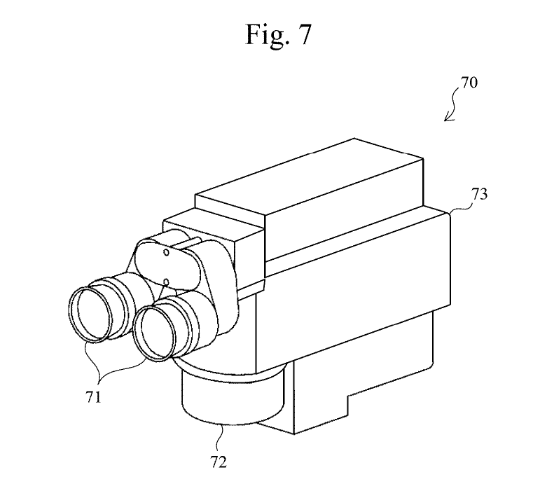

FIG. 7 is a schematic diagram illustrating an example of an appearance of the operating microscope 70 according to the third embodiment. The operating microscope 70 includes eyepieces 71, a light flux receiver 72, and a chassis 73 that houses an optical component constituting various kinds of light paths and a control apparatus that controls various kinds of behaviors. The eyepieces 71 are members that include two eyepiece regions (for example, a first eyepiece and a second eyepiece) including ocular lenses and are looked by the eyes of the user (for example, the operator) for observation of the tissue BT by the user such as the doctor (the operator). The light flux receiver 72 has a configuration serving as an irradiation port to irradiate the tissue BT with an observation light from an operating microscope light source 80 and the excitation light from the fluorescence excitation light source 30 and also serving as a light receiving port to receive a reflected light from the tissue BT. For example, the light flux receiver 72 is a light receiving port (an entrance) from which the reflected light via the tissue BT and the light generated from the tissue BT (for example, the fluorescence) enter.

<Internal Configuration of Operating Microscope>

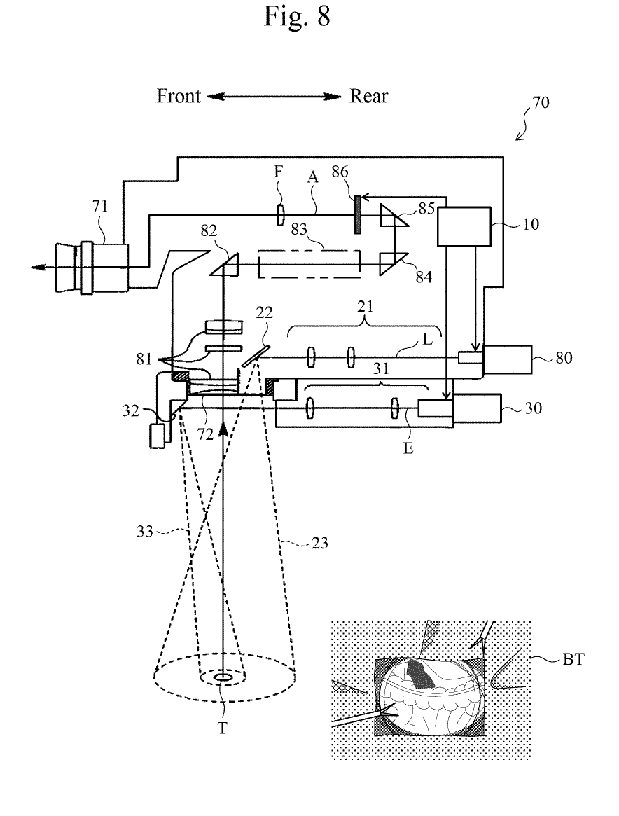

FIG. 8 is a drawing illustrating an example of an internal configuration of the operating microscope 70 according to the third embodiment. As one example, the operating microscope 70 includes the eyepieces 71, the light flux receiver 72, the operating microscope light source 80, the fluorescence excitation light source 30, an illumination light path L that guides an illuminating light from the operating microscope light source 80 to the tissue BT (including the affected part (as one example, the tumor part)) via the light flux receiver 72, an excitation light path E that guides the excitation light from the fluorescence excitation light source 30 to the tissue BT (including the affected part (as one example, the tumor part)) via a mirror 32, an observation light path A that guides the reflected light from the tissue BT and the fluorescence from the affected part (as one example, the tumor part) to the eyepieces 71 via the light flux receiver 72, liquid crystal shutters 86 disposed at the observation light path A, and the control apparatus 10 that controls the behavior timings of the operating microscope light source 80, the fluorescence excitation light source 30, and the liquid crystal shutters 86. While the control apparatus 10 is housed in the chassis 73 in FIG. 8, a configuration in which the control apparatus 10 can be, for example, externally coupled may be employed.

Similar to the first embodiment, while a white LED light source with an illuminance of, for example, 60000 lux, brightness sufficient for surgery, is usable as the operating microscope light source 80, the illumination may have the illuminance of at least, for example, 500 to 1000 lux. The operating microscope light source 80 is coupled to the control apparatus 10 such that the control apparatus 10 controls the light-on and off timings (a light-on time and a light-off time). Although not illustrated, the operating microscope light source 80 includes, for example, a driver. Turning ON and OFF power of an LED in accordance with the light-on and off timings instructed by the control apparatus 10 causes the operating microscope light source 80 to behave so as to repeat light-on and light-off.

Similar to the first embodiment, the fluorescence excitation light source 30 is a light source that emits a light, for example, having a wavelength within a predetermined bandwidth. For example, with the use of the 5-ALA as the fluorescer, as the fluorescence excitation light source 30, a light source with a narrow bandwidth emitting a light (an ultraviolet light) with a wavelength of, for example, 385 nm to 425 nm is usable. Similar to the operating microscope light source 80, the fluorescence excitation light source 30 is coupled to the control apparatus 10 such that the control apparatus 10 controls the light-on and off timings (a light-on time and a light-off time). For example, the fluorescence excitation light source 30 includes, a driver and a controller (not illustrated), turns ON and OFF power of the light source in accordance with the light-on and off timings instructed by the control apparatus 10 causes the fluorescence excitation light source 30 to behave so as to repeat light-on and light-off. The light-on and off timings of the surgery lamp 20 and the light-on and off timings of the fluorescence excitation light source 30 are controlled such that, for example, the light-on and off timings occur in alternation at every predetermined period (for example, 1/60 seconds) or partially overlap with one another (for example, a period during which both of the surgery lamp 20 and the fluorescence excitation light source 30 light on or light off is partially present).

As one example, the illumination light path L includes an illuminating optical system 21 including a plurality of lenses and a mirror 22. The mirror 22, which is located at the latter part of the illuminating optical system 21, changes the light path of the illuminating light that has passed through the illuminating optical system 21 to appropriately irradiate the tissue BT with an illuminating light 23.

As one example, the excitation light path E includes an excitation optical system 31 including a plurality of lenses and the mirror 32. The mirror 32, which is located at the latter part of the excitation optical system 31, changes the light path of the excitation light that has passed through the excitation optical system 31 to appropriately irradiate the tissue BT with an excitation light 33.

As one example, the observation light path A includes objective optical systems 81 disposed at the latter part of the light flux receiver 72, a first prism 82 by which the light that has passed through the objective optical systems 81 is reflected to change the light path, a zoom optical system 83, a second prism 84 by which the light that has passed through the zoom optical system 83 is reflected to change the light path, a third prism 85 by which the light is reflected to change the light path, and a notch filter F that cuts the excitation light (as one example, the ultraviolet light) included in the light from the tissue BT. As described later, when passing through the objective optical systems (light dividers) 81, the light from the tissue BT (the reflected light) is divided into two lights (for example, the two lights corresponding to the two eyepiece regions in the eyepieces 71, a right eye image and a left eye image). Then, the divided two lights (a first divided light, a second divided light) each proceed to a first light path and a second light path of the observation light path A and reach the eyepieces 71. The first light path and the second light path of the observation light path A are light paths disposed between the objective optical systems 81 and the eyepieces 71 and through which the two lights divided by the objective optical systems 81 pass. For example, one light (the first divided light) among the two lights divided by the objective optical systems 81 passes through the first light path of the observation light path A and the other light (the second divided light) passes through the second light path of the observation light path A. The notch filter F is arranged to cut, for example, the excitation light 33, which is unnecessary for the fluorescence observation. In the case where the excitation light 33 is, for example, the ultraviolet light, although the doctor or a similar person (the operator) cannot see the ultraviolet light by the eyes, the exposure of the eyes under the ultraviolet light for a long time is hazardous; therefore, arranging a filter to remove such excitation light is desirable. While FIG. 8 uses the prisms 82 to 85 to change the light paths of the light (the reflected light) from the tissue BT, for example, the prisms 82 to 85 may be simply mirrors.

As one example, the liquid crystal shutters 86 are arranged at the latter part of the first prism 82 and at the previous part of the eyepieces 71 on the observation light path A. The liquid crystal shutters 86 as the eyesight restrictors, for example, include a first eyesight restrictor disposed at the first light path of the observation light path A and a second eyesight restrictor disposed at the second light path of the observation light path A. The liquid crystal shutters 86 are, for example, coupled to the control apparatus 10 such that the control apparatus 10 controls timings at which the first eyesight (for example, the eyesight corresponding to the eyesight of the left eye of the user) at the first eyesight restrictor and the second eyesight (for example, the eyesight corresponding to the eyesight of the right eye of the user) at the second eyesight restrictor are restricted in units of predetermined periods (for example, 1/60 seconds). For example, the liquid crystal (for example, the liquid crystal corresponding to the left eye) of the first eyesight restrictor (for example, the left eye shutter) and the liquid crystal (for example, the liquid crystal corresponding to the right eye) of the second eyesight restrictor (for example, the right eye shutter) of the liquid crystal shutters 86 are switched between the light shielding state and the light transmission state in alternation in units of the predetermined periods.

As one example, the control apparatus 10 transmits the control signal to the operating microscope light source 80, the fluorescence excitation light source 30, and the driver (not illustrated) and the controller (not illustrated) of the liquid crystal shutters 86 to instruct these apparatuses on the light-on timings and the light-off timings of these light sources and the opening and closing timings of the liquid crystal shutters 86. For example, when the above-described first eyesight is in the light shielding state (the left eye shutter closed) and the second eyesight is in the light transmission state (the right eye shutter opened) at the liquid crystal shutters 86, the control apparatus 10 controls the respective behaviors such that the operating microscope light source 80 lights off and the fluorescence excitation light source 30 lights on. Additionally, for example, when the above-described first eyesight is in the light transmission state (the left eye shutter opened) and the second eyesight is in the light shielding state (the right eye shutter closed), the control apparatus 10 controls the respective behaviors such that the operating microscope light source 80 lights on and the fluorescence excitation light source 30 lights off.

<Optical System Configuration>

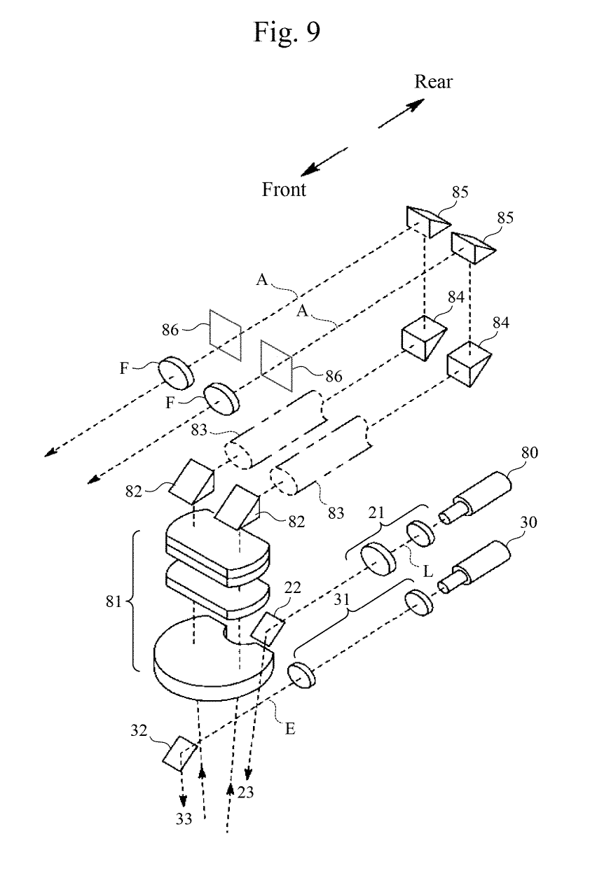

FIG. 9 is a drawing illustrating a configuration example of an optical system of the operating microscope 70 according to the third embodiment. FIG. 9 illustrates only the optical system configuration and omits the control apparatus 10 and the eyepieces 71.

The light-on and light-off of the illuminating light from the operating microscope light source 80 and the excitation light from the fluorescence excitation light source 30 and the opening and closing of the right and left eye shutters on the liquid crystal shutters 86 are controlled at the timings illustrated in FIG. 3. For example, the operating microscope light source 80 is lit on for a predetermined period (for example, 1/60 seconds) and the tissue BT is irradiated with the illuminating light irradiated from the operating microscope light source 80 via the illuminating optical system 21 and the mirror 22. The reflected light from the tissue BT, for example, becomes the two lights corresponding to the right and left eyes observed at the eyepieces 71, and the two lights are introduced to the liquid crystal shutters 86 via the objective optical systems 81, the first prism 82, the zoom optical system 83, the second prism 84, and the third prism 85. In the case where the operating microscope light source 80 lights on, the liquid crystal shutters 86 are, for example, controlled such that the first eyesight (for example, the eyesight at the left eye shutter) enters the light transmission state and the second eyesight (for example, the eyesight at the right eye shutter) enters the light shielding state. Therefore, the reflected light from the tissue BT passes through only the first eyesight restrictor (the left eye shutter) and reaches only the one eyepiece region (for example, the first eyepiece including the left eye ocular lens) of the eyepieces 71 via, for example, the notch filter F.

When the predetermined period (for example, the above-described 1/60 seconds) passes from when the operating microscope light source 80 is lit on, the control is performed such that the operating microscope light source 80 lights off and the fluorescence excitation light source 30 lights on for the predetermined period (for example, 1/60 seconds). Additionally, the control is performed such that the first eyesight restrictor (the left eye shutter) of the liquid crystal shutters 86 enters the light shielding state and the second eyesight restrictor (the right eye shutter) enters the light transmission state. During this period, the tissue BT is irradiated with the excitation light from the fluorescence excitation light source 30 via, for example, the excitation optical system 31 and the mirror 32. The irradiation of the tissue BT with the excitation light 33 emits the fluorescence from the affected part (for example, the tumor part) of the tissue BT. This fluorescence from the affected part, for example, becomes two lights corresponding to the right and left eyes observed at the eyepieces 71, and the two lights are introduced to the liquid crystal shutters 86 via the objective optical systems 81, the first prism 82, the zoom optical system 83, the second prism 84, and the third prism 85. In the case where the fluorescence excitation light source 30 lights on, as described above, since the liquid crystal shutters 86 are controlled such that the first eyesight restrictor (the left eye shutter) enters the light shielding state and the second eyesight restrictor (the right eye shutter) enters the light transmission state, the fluorescence passes through only the second eyesight restrictor (the right eye shutter). Further, since the reflected light of the excitation light via the tissue BT is removed by, for example, the notch filter F, only the fluorescence reaches the other eyepiece region (for example, the second eyepiece including the right eye ocular lens) of the eyepieces 71.

The above-described processes are switchingly repeated at every predetermined period (for example, 1/60 seconds). When the operating microscope light source 80 lights on (the fluorescence excitation light source 30 lights off) and the left eye shutter is in the light transmission state (the right eye shutter is in the light shielding state), the doctor or a similar person (the operator) who looks through the eyepieces 71 can brightly observe the tissue BT by the left eye. Meanwhile, when the fluorescence excitation light source 30 lights on (the surgery lamp 20 lights off) and the right eye shutter is in the light transmission state (the left eye shutter is in the light shielding state), the doctor or a similar person (the operator) who looks through the eyepieces 71 can observe the tumor part (the affected part) emitting the fluorescence in the dark eyesight by the right eye. By switchingly controlling these states at every constant period (for example, every 1/60 seconds), the image of the right eye and the image of the left eye are seen to be combined in a pseudo manner in the brain of the doctor or a similar person (the operator), and thus the fluorescence from the tumor part (the affected part) of the tissue BT is seen to be highlighted (The fluorescence from the tissue BT is not negated by the operating microscope light source 80 but can be clearly seen).

While this embodiment synchronizes the operating microscope light source 80 with the left eye shutter on the liquid crystal shutters 86 and synchronizes the fluorescence excitation light source 30 with the right eye shutter on the liquid crystal shutters 86, the synchronization timing may be reversed. For example, while this embodiment uses the light source that can perform a coaxial illumination on the tissue BT as the operating microscope light source 80, a light source that can perform a ring illumination on the tissue BT may be used. For example, with the use of the ring illumination, the operating microscope light source 80 according to this embodiment can evenly irradiate the tissue BT from all directions.

<Details of Synchronization Timing Control and Process Contents>

The controls of the light-on and light-off timings of the operating microscope light source 80 and the fluorescence excitation light source 30 and the opening and closing timings of the liquid crystal shutters of the third embodiment are, for example, as shown by the timing chart of FIG. 3 and its description; therefore, the detailed description is omitted here. Note that in FIG. 3 and its description, “the surgery lamp” can be read as “the operating microscope light source” and “the liquid crystal glasses” as “the liquid crystal shutters.”

The control process contents by the control apparatus 10 in the third embodiment are, for example, as illustrated in the flowchart of FIG. 4 and its description; therefore, the detailed description is omitted here. Note that in FIG. 4 and its description, “the surgery lamp” can be read as “the operating microscope light source” and “the liquid crystal glasses” as “the liquid crystal shutters.” Moreover, while the first embodiment is described that the control apparatus 10 and the liquid crystal glasses 40 perform the wireless communications, in the third embodiment, for example, the control apparatus 10 and the liquid crystal shutters 86 are coupled by wires for ensuring communications (Note that this does not deny a possibility of wireless communications).

The third embodiment is an application of the principle according to the first embodiment to the operating microscope. While this embodiment refers to the microscope as “the operating microscope,” the operating microscope is not limited to the surgery application and is widely applicable to the general medical practice. The operating microscope 70 includes a light introduction mechanism (for example, the illumination light path L and the excitation light path E), the eyepieces 71, a usual light path (the observation light path A), the light dividers (the objective optical systems 81), the eyesight restrictors (for example, the liquid crystal shutters 86), and the control apparatus 10. The light introduction mechanism introduces the first light and the second light emitted from the light sources (for example, the operating microscope light source 80 and the fluorescence excitation light source 30) to the biological tissue. The usual light path guides the light from the biological tissue to the eyepieces 71. The light dividers, which are disposed on the usual light path, divide the light from the biological tissue into lights of the right and left eyesights of the user. The eyesight restrictors are disposed between the light dividers and the eyepieces. The control apparatus 10 controls the behaviors of the light sources and the behaviors of the eyesight restrictors. The control apparatus 10 performs the open/close control on the first eyesight and the second eyesight (the eyesight of the left eye can be defined as the first eyesight and the eyesight of the right eye as the second eyesight, or conversely the eyesight of the right eye can be defined as the first eyesight and the eyesight of the left eye as the second eyesight) of the eyesight restrictors in alternation at every predetermined time interval. Then, the control apparatus 10 configures the first light irradiation and the second light irradiation so as to be performed in alternation, matches the opening and closing timings of the first eyesight with the timings of introduction/non-introduction of the first light, and matches the opening and closing timings of the second eyesight with the timings of the introduction/non-introduction of the second light. The operating microscope 70 according to this embodiment brightly irradiates the biological tissue, thereby ensuring the surgery while directly confirming the tumor part (the affected part). In the respective embodiments, for example, the second light is not only the fluorescence but may be a phosphorescence, and the second light may be a light emitted from the tissue BT by the light irradiation to the tissue BT.

The light-on and light-off timings (the first light irradiation timing and the second light irradiation timing) of the operating microscope light source 80 and the fluorescence excitation light source 30 may occur in alternation as illustrated in FIG. 3 or may partially overlap with one another.

As described above, the control apparatus 10 communicates with the eyesight restrictors (for example, the liquid crystal shutters 86) to perform the open/close control on the eyesight of the left eye and the eyesight of the right eye. The opening and closing timings of the eyesight of the left eye and the eyesight of the right eye at these eyesight restrictors, for example, may be controlled so as to occur in alternation as described above or may partially overlap with one another as another configuration. The case where the timings partially overlap with one another can include, for example, the case where there are time slots in which both of the eyesight of the right eye and the eyesight of the left eye are in the open state and the closed state, or there is a time slot in which both eyesights are in the closed state or the open state.

### (4) Fourth Embodiment

A surgery assistance system according to the fourth embodiment is equivalent to a modification of the surgery assistance system according to the first embodiment. For example, while the first embodiment controls the surgery lamp 20 and the fluorescence excitation light source 30 to shift the timings such that the surgery lamp 20 and the fluorescence excitation light source 30 each light on and lights off at every predetermined time interval (for example, every 1/60 seconds), the fourth embodiment performs a control in which the light amount (or the illuminance) of the surgery lamp 20 and the light amount (or the illuminance) of the fluorescence excitation light source 30 are each changed with the timings shifted at every predetermined time interval (for example, every 1/60 seconds). The fourth embodiment is similar to the first embodiment in the configuration of the surgery assistance system and processes other than the control of the light amount (or the illuminance) of the surgery lamp 20 and the control of the light amount (or the illuminance) of the fluorescence excitation light source 30. The following describes the fourth embodiment.

<Configuration of Surgery Assistance System>

The configuration similar to that of the surgery assistance system 1 according to the first embodiment is employable as a hardware configuration of the surgery assistance system according to the fourth embodiment. Meanwhile, the software configuration differs as described below.

<Process Outline>

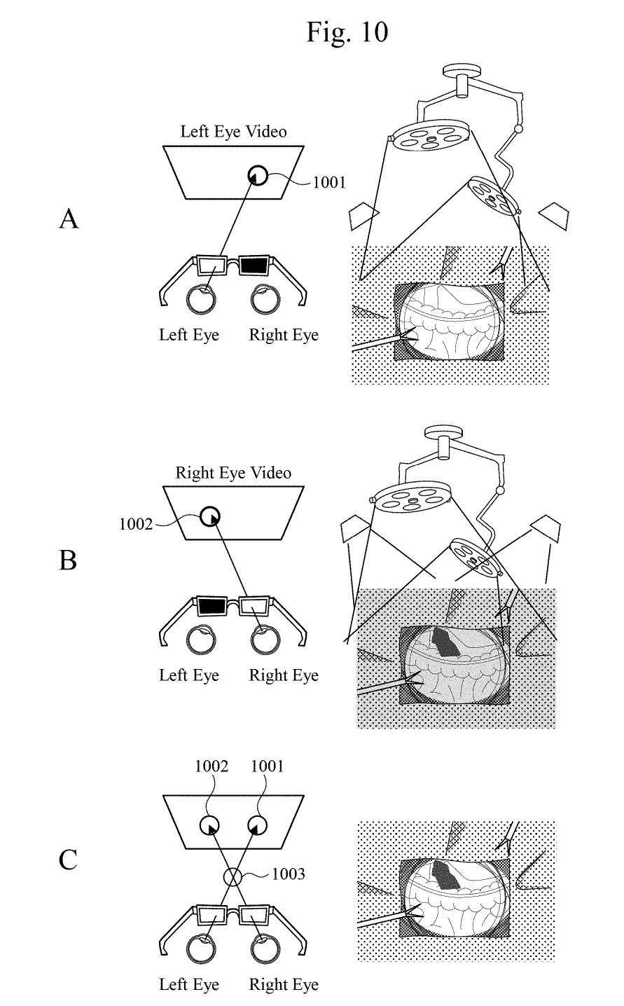

FIG. 10 includes drawings describing an outline of the light irradiation timing control. Here illustrates an example in which the light irradiation by the surgery lamp 20 and the irradiation of the excitation light by the fluorescence excitation light source 30 are performed in alternation. FIG. 10(A) illustrates a left eye video (an odd number field) 1001 seen by the doctor or a similar person who wears the liquid crystal glasses 40 through the left eye shutter (the left eyesight) in the light transmission state for 1/60 seconds during which the surgery lamp 20 lights on at a predetermined light amount (may be a predetermined illuminance, for example, may be the maximum light amount (or the maximum illuminance) that can be output from the surgery lamp 20). The light amount of the excitation light from the fluorescence excitation light source 30 at this time is controlled to be, for example, a predetermined light amount (For example, the light amount can be set to 5%, 10%, and 30% with respect to the maximum light amount, and the light amount can be relatively lower than that of the surgery lamp. Alternatively, for example, the fluorescence excitation light source 30 may be lit off) smaller than the maximum light amount that can be output from the fluorescence excitation light source 30.

FIG. 10(B) illustrates a right eye video (an even number field) 1002 seen by the doctor or a similar person who wears the liquid crystal glasses 40 through the right eye shutter (the right eyesight) in the light transmission state for 1/60 seconds during which the light amount of the surgery lamp 20 is, for example, reduced (or the illuminance is lowered) to less than the above-described predetermined light amount and the surgery lamp 20 lights on, which is for the 1/60 seconds during which the light amount of the excitation light from the fluorescence excitation light source 30 is increased (the increased predetermined light amount (for example, the maximum light amount can be employed)) to more than the light amount for 1/60 seconds immediately before and the tissue BT is irradiated with the excitation light. FIG. 10(C) illustrates an image 1003 combined in the brain of the doctor or a similar person who wears the liquid crystal glasses 40.

The surgery lamp 20 and the fluorescence excitation light source 30 can include, for example, LEDs in respective colors (LEDs with various kinds of wavelengths). In this case, the light amount of the surgery lamp 20 and the light amount of the excitation light from the fluorescence excitation light source 30 can be controlled by controlling (for example, changing a resistance value by a variable resistor) a current value flowing through the surgery lamp 20 and the fluorescence excitation light source 30. Alternatively, the light amount of the surgery lamp 20 and the light amount of the excitation light from the fluorescence excitation light source 30 can be controlled by adjusting a duty ratio of a PWM signal indicative of the light-on time corresponding to the light amount. Although the former changes the color of the LED light by reducing the light amount, the latter is advantageous in that only the light amount decreases and the color of the LED light becomes constant.

As illustrated in FIG. 10, the control apparatus 10 controls the liquid crystal shutters on the liquid crystal glasses 40 such that the openings/closings of the left eye shutter (the eyesight of the left eye (can also be referred to as the first eyesight)) and the right eye shutter (the eyesight of the right eye (can also be referred to as the second eyesight)) are switched in alternation in units of, for example, 1/60 seconds (see FIG. 10(A) and FIG. 10(B)). As one example, with the left eye shutter open, the surgery lamp 20 is controlled such that the surgery lamp 20 lights on at, for example, the maximum light amount (or the maximum illuminance) and the tissue BT is irradiated with the illuminating light (for example, the white LED light), and the fluorescence excitation light source 30 is controlled so as to output the excitation light at, for example, the light amount (may light off) at a predetermined proportion with respect to the maximum light amount. For example, an image captured by the left eye constitutes an image 1001 in the odd number field and an image captured by the right eye constitutes an image 1002 in the even number field. For example, the image of the right eye and the image of the left eye are seen to be combined in a pseudo manner, and thus the fluorescence from the tissue BT is seen to be highlighted (see FIG. 10(C)). The image (the combined image) combined in the brain of the doctor or a similar person becomes a frame image (a played image) 1003 in units of 1/30.

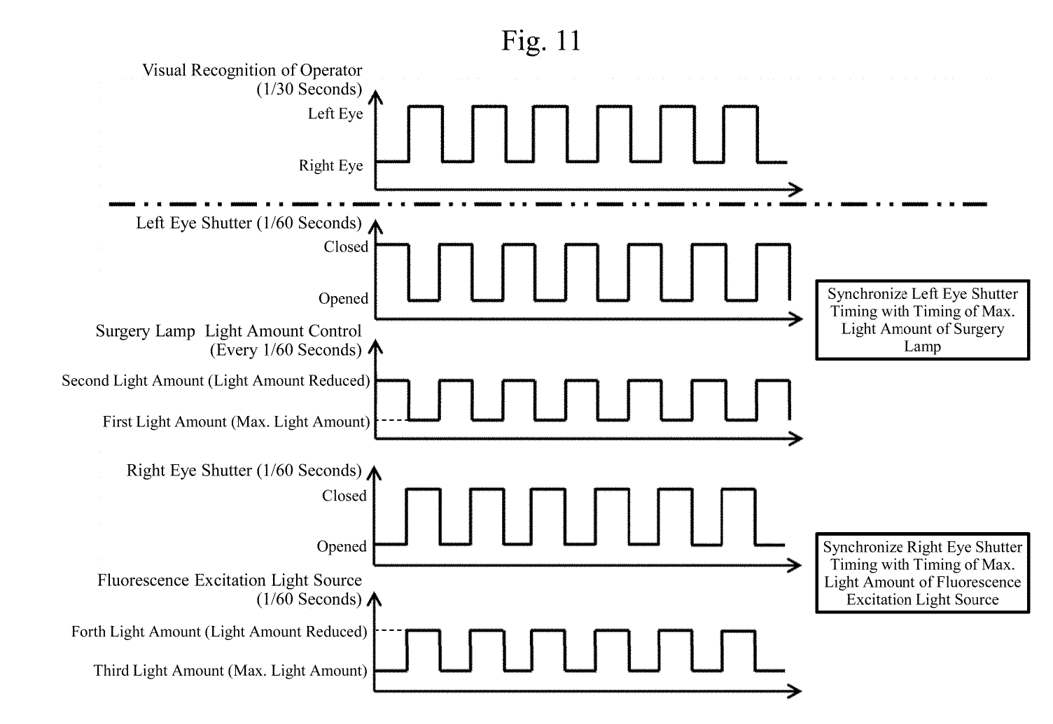

FIG. 11 is a timing chart illustrating switching timings to open/close the liquid crystal shutters on the liquid crystal glasses 40, a light amount control timing of the surgery lamp 20, and a light amount control timing of the fluorescence excitation light source 30. While FIG. 11 illustrates the case where the light amount control timing of the surgery lamp 20 and the light amount control timing of the fluorescence excitation light source 30 occur in alternation as one example, the light amount control timing of the surgery lamp 20 may partially overlap with the light amount control timing of the fluorescence excitation light source 30.

In FIG. 11, for example, the control apparatus 10 performs wireless communications with the liquid crystal glasses 40 and controls the opening and closing of the liquid crystal shutters in accordance with this timing chart. As illustrated in FIG. 11, the left eye shutter repeats the opening and closing at every 1/60 seconds. While the right eye shutter repeats the opening and closing at every 1/60 seconds as well, the opening and closing timings are opposite to those of the left eye shutter. While the left eye shutter is open (while the right eye shutter is closed), the surgery lamp 20 is turned ON (bright) at, for example, a first light amount (for example, the maximum light amount (or the maximum illuminance)), and while the left eye shutter is closed (while the right eye shutter is open), the light amount of the surgery lamp 20 is reduced to the second light amount, which is smaller than the first light amount. How extent that the light amount of the surgery lamp 20 is reduced may be configured to be appropriately set or adjusted by the doctor or a similar person (the operator). While the right eye shutter is open (while the left eye shutter is closed), the fluorescence excitation light source 30 is turned ON (bright) at, for example, a third light amount (for example, the maximum light amount (or the maximum illuminance)), and while the right eye shutter is closed (while the left eye shutter is open), for example, the light amount of the fluorescence excitation light source 30 is reduced (dark) to a fourth light amount (for example, may be turned OFF), which is smaller than the third light amount. How extent that the light amount of the fluorescence excitation light source 30 is reduced may be configured to be appropriately set or adjusted by the doctor or a similar person (the operator).

For example, the control is performed such that the opening and closing timings of the left eye shutter (the first eyesight, the eyesight of the left eye) become identical to the timing of light amount control of the surgery lamp 20 and the opening and closing timings of the right eye shutter (the second eyesight, the eyesight of the right eye) become identical to the timing of light amount control of the excitation light from the fluorescence excitation light source 30. As one example, in the case where the surgery lamp 20 lights on (the fluorescence excitation light source 30 lights on (for example, may light off), for example, at the fourth light amount (reduced light amount)) at the first light amount (for example, the maximum light amount (or the maximum illuminance)) and the left eye shutter is open, which is the light transmission state, the light (the illuminating light at the maximum light amount) from the surgery lamp 20 irradiating the tissue BT transmits the left eye shutter. This allows the doctor or a similar person (the operator) who wears the liquid crystal glasses 40 to observe the tissue BT brightly by the left eye. Meanwhile, when the fluorescence excitation light source 30 lights on (the light amount of the surgery lamp 20 is reduced) at, for example, the third light amount and the right eye shutter is open, which is the light transmission state, the tissue BT is irradiated with the excitation light while the surgery lamp 20 whose light amount has been reduced irradiates the tissue BT. This causes the fluorescence generated from the tissue BT and the light (the illuminating light whose light amount has been reduced) from the surgery lamp 20 that has irradiated the tissue BT transmit the right eye shutter. Accordingly, the doctor or a similar person (the operator) who wears the liquid crystal glasses 40 can observe the tumor part (the affected part) emitting the fluorescence in the eyesight with a difference between bright and dark (a difference between bright and dark of the surgery lamp 20 with the fluorescence). By switchingly controlling these states at every constant period (for example, every 1/60 seconds), the image of the right eye and the image of the left eye are seen to be combined in a pseudo manner in the brain of the doctor or a similar person (the operator), and thus the fluorescence from the tumor part (the affected part) of the tissue BT is seen to be highlighted (The fluorescence from the tissue BT is not negated by the surgery lamp 20 but can be clearly seen). In this embodiment, the light amount of the surgery lamp 20 and the light amount of the fluorescence excitation light source 30 are controlled (adjusted) as one example. This reduces the difference between bright and dark of the surgery lamp 20 with the fluorescence compared with the case where the surgery lamp 20 and the fluorescence excitation light source 30 are lit off and allows the operator to easily see both an operative field and the fluorescence. While in the timing chart of FIG. 3, the light amount control is performed such that the light amount of the surgery lamp 20 and the light amount of the fluorescence excitation light source 30 are equally reduced at a constant proportion, this should not be constructed in a limiting sense. For example, the proportion of the light amount adjustment may be changed at every predetermined time interval (for example, every 1/60 seconds). Further, for example, the doctor or a similar person (the operator) may be able to appropriately set the proportion of the light amount adjustment using the input apparatus 102. This allows this operator to achieve an optimal difference between bright and dark.

The control apparatus 10 may perform the control so as to match the opening and closing timings of the left eye shutter (the first eyesight, the eyesight of the left eye) with the timing of light amount control of the fluorescence excitation light source 30 and match the opening and closing timings of the right eye shutter (the second eyesight, the eyesight of the right eye) with the timing of light amount control of the surgery lamp 20 through communications with the surgery lamp 20, the fluorescence excitation light source 30, and the liquid crystal glasses 40.

The roles of the left eye shutter and the right eye shutter may be switched at a predetermined timing. For example, the control apparatus 10 may perform the control so as to match the opening and closing timings of the left eye shutter with the timing of light amount control of the fluorescence excitation light source 30 and match the opening and closing timings of the right eye shutter with the timing of light amount control of the surgery lamp 20 for a constant period through communications with the liquid crystal glasses 40, the surgery lamp 20, and the fluorescence excitation light source 30. The control apparatus 10 may perform the control so as to match the opening and closing timings of the left eye shutter with the timing of light amount control of the surgery lamp 20 and match the opening and closing timings of the right eye shutter with the timing of light amount control of the fluorescence excitation light source 30, for example, after a lapse of a constant period. For example, the control apparatus 10 repeats this control to switch the roles. This configuration ensures avoiding a problem of, for example, losing a sense of perspective caused by seeing the tissue BT only by one eye of the wearing target.

<Process Contents by Control Apparatus: Contents of Light Irradiation Control Process by Illumination Control Program>

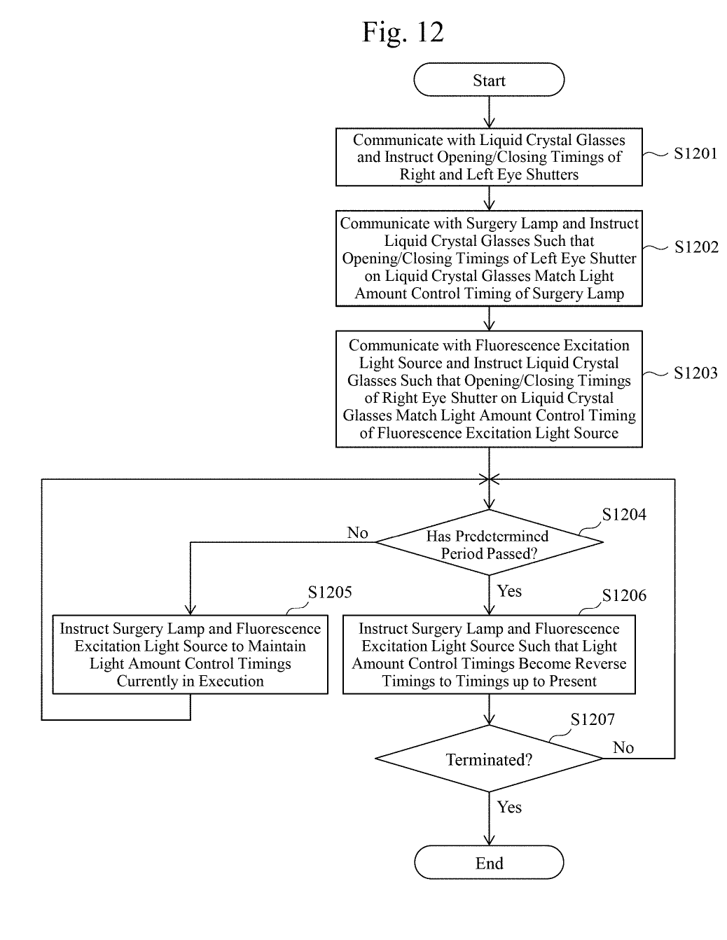

FIG. 12 is a flowchart describing process contents (contents of the illumination control program 1061) of the control apparatus 10 according to the fourth embodiment. The controller 101 in the control apparatus 10, for example, reads the illumination control program 1061 from a memory (for example, a Read Only Memory (ROM)) into an internal memory (not illustrated) disposed inside the controller 101 to perform the illumination control program 1061. For example, the controller 101 performs respective steps as follows in accordance with this illumination control program 1061.

### (i) Step 1201

For example, the wearing target, such as the doctor (the operator) who wears the liquid crystal glasses 40 and is going to perform a surgery, turns ON power of the liquid crystal glasses 40, turns ON power of the control apparatus 10, and sets a mode to a surgery mode. Then, the controller 101 in the control apparatus 10 starts communicating with the liquid crystal glasses 40 and instructs the liquid crystal glasses 40 such that the opening and closing timings of the left eye shutter and the right eye shutter on the liquid crystal glasses 40 become the timings illustrated in FIG. 11 by transmission of the control signal.

The liquid crystal glasses 40 sequentially open/close the left eye shutter and the right eye shutter in accordance with the opening and closing timings of the shutters received from the control apparatus 10. The right and left eye shutters on the liquid crystal glasses 40 are opened/closed at, for example, every 1/60 seconds in alternation (see FIG. 11).

### (ii) Step 1202

The controller 101 communicates with the surgery lamp 20 and instructs the liquid crystal glasses 40 such that the opening and closing timings of the left eye shutter on the liquid crystal glasses 40 match the timing of light amount control (the light amount adjustment) of the surgery lamp 20 by transmission of the control signal.

The surgery lamp 20 behaves the driver or the controller (not illustrated) in accordance with the timing of light amount control (the light amount adjustment) notified from the control apparatus 10 to control the light amount (or the illuminance) of the white LED according to the opening and closing timings of the left eye shutter on the liquid crystal glasses 40. Similar to the left eye shutter on the liquid crystal glasses 40, the timing of light amount control (the light amount adjustment) of the white LED is, for example, every 1/60 seconds. For example, regarding the surgery lamp 20, a period during which the tissue BT is irradiated at the first light amount (for example, the maximum light amount (or the maximum illuminance)) and a period during which the light amount is reduced (or the illuminance is lowered) to the second light amount and the tissue BT is irradiated are switched in alternation at, for example, every 1/60 seconds. Regarding the first light amount and the second light amount, the maximum light amount and the reduced light amount are used as the example. However, any light amount, not the maximum light amount, can be set as the light amount and the light amount control may use the light amount further reduced from any of the light amount. For example, any light amount can be set as the light amount controlled during the above-described respective light amount control periods (for example, 1/60 seconds). For example, the light amount of the surgery lamp 20 may be controlled so as to be sequentially changed at the respective periods as follows. The surgery lamp 20 lights on at the maximum light amount during a first period (for example, 1/60 seconds), lights on at the light amount 40% of the maximum light amount during the next period, lights on at the light amount 90% of the maximum light amount during yet next period, and lights off during yet next period. Furthermore, for example, during surgery, the operator (for example, the doctor) may use the input apparatus 102 to input the instruction to change the light amount during each period (for example, each period of 1/60 seconds), and the controller 101 may control the light amount of the surgery lamp 20 in response to the instruction. This ensures providing a surgery environment suitable for a sensation of each operator.

(iii) Step 1203

The controller 101 communicates with the fluorescence excitation light source 30 and instructs the liquid crystal glasses 40 such that the opening and closing timings of the right eye shutter on the liquid crystal glasses 40 match the timing of light amount control (the light amount adjustment) of the fluorescence excitation light source 30 by transmission of the control signal.

The fluorescence excitation light source 30 behaves the driver or the controller (not illustrated) in accordance with the timing of light amount control (the light amount adjustment) notified from the control apparatus 10 to control the light amount (or the illuminance) of an LED with a predetermined wavelength according to the opening and closing timings of the right eye shutter on the liquid crystal glasses 40. For example, similar to the right eye shutter on the liquid crystal glasses 40, the timing of light amount control (the light amount adjustment) of the LED with the predetermined wavelength is, for example, every 1/60 seconds. For example, regarding the fluorescence excitation light source 30, a period during which the tissue BT is irradiated at the third light amount (for example, the maximum light amount (or the maximum illuminance)) and a period during which the light amount is reduced (or the illuminance is lowered) to the fourth light amount and the tissue BT is irradiated are switched in alternation at, for example, every 1/60 seconds. Regarding the third light amount and the fourth light amount, the maximum light amount and the reduced light amount are used as the example. However, any light amount, not the maximum light amount, can be set as the light amount and the light amount control may use the light amount further reduced from any of the light amount. For example, any light amount can be set as the light amount controlled during the above-described respective light amount control periods (for example, 1/60 seconds). For example, the light amount of the fluorescence excitation light source 30 may be controlled so as to be sequentially changed at the respective periods as follows. The fluorescence excitation light source 30 lights on at the maximum light amount during a first period (for example, 1/60 seconds), lights on at the light amount 40% of the maximum light amount during the next period, lights on at the light amount 90% of the maximum light amount during yet next period, and lights off during yet next period. Furthermore, for example, during surgery, the operator (for example, the doctor) may use the input apparatus 102 to input the instruction to change the light amount during each period (for example, each period of 1/60 seconds), and the controller 101 may control the light amount of the fluorescence excitation light source 30 in response to the instruction. This ensures providing a surgery environment suitable for a sensation of each operator.

### (iv) Step 1204

The controller 101 determines whether a predetermined period (for example, may be set to one minute. The setting period is changeable by the user.) has passed from a start of a process of timing synchronization between the surgery lamp 20, the fluorescence excitation light source 30, and the liquid crystal glasses 40 or not. When the predetermined period has passed (Yes at Step 1204), the process by the controller 101 transitions to Step 1206. When the predetermined period has not passed (No at Step 1204), the process by the controller 101 transitions to Step 1205.

### (v) Step 1205

The controller 101 instructs the respective surgery lamp 20 and fluorescence excitation light source 30 to continue the behaviors at the light amount control timing currently in execution.

For example, the surgery lamp 20 receives this instruction to continue the behavior at the light amount control timing currently in execution from the control apparatus 10 to continue the behavior in accordance with the instruction. For example, the fluorescence excitation light source 30 also receives this instruction to continue the behavior at the light amount control timing currently in execution from the control apparatus 10 to continue the behavior in accordance with the instruction.

### (vi) Step 1206

For example, the controller 101 instructs the respective surgery lamp 20 and fluorescence excitation light source 30 such that the light amount control timing up to the present becomes the reverse timing to the timing up to the present by transmission of the control signals. Since the opening and closing timings of the shutters on the liquid crystal glasses 40 are identical to those up to the present, the opening and closing timings are not especially instructed.

For example, in the case where the light amount control timing of the surgery lamp 20 has matched the opening and closing timings of the left eye shutter on the liquid crystal glasses 40 and the light amount control timing of the fluorescence excitation light source 30 has matched the opening and closing timings of the right eye shutter on the liquid crystal glasses 40 up to the present, the controller 101 instructs the surgery lamp 20 and the fluorescence excitation light source 30 such that the light amount control timing of the surgery lamp 20 matches the opening and closing timings of the right eye shutter and the light amount control timing of the fluorescence excitation light source 30 matches the opening and closing timings of the left eye shutter by transmission of the control signal.

For example, the surgery lamp 20 receives the instruction (the control signal) to change the timing from the control apparatus 10 and changes the light amount control timing in accordance with the instruction. For example, the fluorescence excitation light source 30 receives the instruction (the control signal) to change the timing from the control apparatus 10 and changes the light amount control timing in accordance with the instruction.

(vii) Step 1207

The controller 101, for example, determines whether the user (the doctor or a similar person as the operator) has input an instruction (a termination signal) to terminate the process from the input apparatus 102 or not. For example, when the instruction to terminate the process has not been input (No at Step 1207), the process transitions to Step 1204 to continue the process. For example, when the instruction to terminate the process has been input (Yes at Step 1207), the light irradiation control process by this illumination control program 1061 is terminated.

While FIG. 12 represents the sequential execution of the processes of Steps 1201 to 1203 as one example, for example, the processes of these steps may be performed almost simultaneously. For example, the processes of Step 1204 and Step 1205 are not essential, and Step 1206 may be performed after Step 1203. When the process is not terminated, for example, the similar behaviors are continued at the surgery lamp 20, the fluorescence excitation light source 30, and the liquid crystal glasses 40.

In the fourth embodiment, the control apparatus 10 performs an open/close control on the eyesight of the left eye and the eyesight of the right eye of the wearing target of the liquid crystal glasses (the eyesight restricting apparatus) 40 in alternation at every predetermined time interval and controls these behaviors such that the timing of the light amount control of the light (the first light) and the timing of the light amount control of the excitation light (the second light) by the light sources (the surgery lamp 20 and the fluorescence excitation light source 30) differ. For example, the light amount control timing of the first light and the light amount control timing of the second light may occur in alternation or may partially overlap with one another. For example, when the excitation light is irradiated at the third light amount, the first light is irradiated at the second light amount smaller than the first light amount. When the excitation light is irradiated at the fourth light amount whose light amount is smaller than the third light amount, the first light is irradiated at the first light amount. The control apparatus 10 controls the light irradiation behaviors such that the opening and closing timings of any one of the first eyesight (for example, equivalent to the eyesight of the left eye) of the first eyesight restrictor and the second eyesight (for example, equivalent to the eyesight of the right eye) of the second eyesight restrictor match the switching control timing of the irradiation by the surgery lamp 20 at the first light amount and the irradiation by the surgery lamp 20 at the second light amount; and the opening and closing timings of the eyesight (for example, the second eyesight, the eyesight of the right eye) other than the eyesight (for example, the first eyesight, the eyesight of the left eye) matched with the switching control timing of the light amounts of the irradiation by the surgery lamp 20 match the timing of the light amount control of the fluorescence excitation light source 30. When the eyesight matched with the switching control timing of the light amounts of the irradiation by the surgery lamp 20 is the eyesight of the right eye, the eyesight other than the above-described eyesight becomes the eyesight of the left eye. For example, it can be expressed that the control apparatus 10 performs the open/close control on the first eyesight and the second eyesight of the liquid crystal glasses (the eyesight restricting apparatus) 40 in alternation at every predetermined time interval and controls these behaviors such that the irradiation of the light (the first light) at the first light amount by the surgery lamp 20 and the irradiation of the excitation light (the second light) at the third light amount by the fluorescence excitation light source 30 occur in alternation (The excitation light at the third light amount is irradiated simultaneously with the irradiation of the first light at the second light amount). In this case, the control apparatus 10 controls the light irradiation behaviors such that the opening and closing timings of the first eyesight match the timing of light amount control of the irradiation of the surgery lamp 20; and the opening and closing timings of the second eyesight match the timing of the light amount control of the fluorescence excitation light source 30. For example, as the surgery lamp 20, the light source (for example, an LED light source emitting a white light) emitting visible light is usable, and as the fluorescence excitation light source 30, the excitation light source (For example, an LED emitting excitation light at a predetermined wavelength is usable.) emitting the excitation light at a predetermined wavelength is usable. In this embodiment, the light emission timings of the visible light (the first light) at the first light amount by the surgery lamp and the excitation light (the second light) at the third light amount by the excitation light source are different. For example, the light emission timings may occur at cycles of mutually emitting the lights in alternation, may occur at cycles in phases opposite to one another, or may occur at cycles of partially overlapping with one another. The light emission timings of the visible light (the first light) at the second light amount by the surgery lamp and the excitation light (the second light) at the fourth light amount by the excitation light source are also different. For example, the light emission timings may occur at cycles of mutually emitting the lights in alternation, may occur at cycles in phases opposite to one another, or may occur at cycles of partially overlapping with one another. According to this embodiment, the user (the doctor or a similar person) needs not to observe the fluorescence emitted from a biological tissue in the dark periphery, ensuring the surgery while directly confirming the tumor part in the bright operating room. According to this embodiment, the biological tissue is irradiated with the visible light at the second light amount while the excitation light is emitted. This reduces the difference between bright and dark between the surgery lamp and the fluorescence, thereby ensuring easily seeing both the operative field and the fluorescence. Note that the light sources (as described above, the one light source may switchingly irradiate the visible light and the excitation light) according to this embodiment each irradiate the biological tissue to which the fluorescer has been administrated with the excitation light and the visible light. According to this embodiment, the irradiation of the excitation light emits the fluorescence from the tumor part of the biological tissue, thus allowing the user to observe the affected part (the generated fluorescence) in the bright room.

As the excitation light emitted by the fluorescence excitation light source 30, the ultraviolet light with the wavelength of, for example, 385 nm to 425 nm is usable. The ultraviolet light cannot be seen by the eyes, and therefore this ensures preventing the excitation light from irradiating not only the affected part but also the entire biological tissue (for example, an organ) with color and hindering the medical practice.

As described in this embodiment, as the eyesight restricting apparatus, the liquid crystal glasses 40, for example, that can switch the right and left eyesights between the transmission and the light shielding in alternation is usable. Besides, the liquid crystal glasses 40 may have the shape like ski goggles. The use of the glasses-shaped and the ski-goggle-shaped apparatuses facilitates wearing the eyesight restricting apparatus by the doctor and the operator, and the wearing of this apparatus does not hinder the medical practice. The eyesight restricting apparatus (or the eyesight restrictors) according to this embodiment, for example, may be configured to include an optical member such as a wavelength filter (for example, a visible filter, a bandpass filter, and a dichroic mirror), which has a function that can control the eyesights of the user by controlling (or restricting) the transmitting light and selects the wavelength of the transmitting light; and a filter (for example, an ND filter and a polarization filter), which adjusts (for example, dims the transmission light) the light amount of the transmitting light.

The control apparatus 10 performs the wireless communications with the eyesight restricting apparatus (for example, the liquid crystal glasses 40) to perform the open/close control on the eyesight of the left eye and the eyesight of the right eye. The opening and closing timings of the eyesight of the left eye and the eyesight of the right eye in this eyesight restricting apparatus, for example, may be controlled so as to occur in alternation or may partially overlap with one another. The case where the timings partially overlap with one another can include, for example, the case where there are time slots in which both of the eyesight of the right eye and the eyesight of the left eye are in the open state and the closed state, or there is a time slot in which both eyesights are in the closed state or the open state. For example, although not illustrated, the liquid crystal glasses 40 at least include a communication apparatus to communicate with the control apparatus 10, a processor that performs an instruction from the control apparatus 10, and a power supply to drive these apparatuses. Since the control apparatus 10 and the eyesight restricting apparatus (the liquid crystal glasses 40) can communicate with one another by wireless communications, the doctor and the operator who wear the eyesight restricting apparatus can smoothly move, and this eliminates the interference to the medical practice such as the surgery due to wiring and the like.

### (5) Fifth Embodiment

For example, while the control apparatus 10 instructs the surgery lamp 20, the fluorescence excitation light source 30, and the liquid crystal glasses 40 on the respective behavior timings in the fourth embodiment, switching ON the liquid crystal glasses 40 autonomously opens/closes the left eye shutter and the right eye shutter at predetermined timings in the fifth embodiment. Through communications with the liquid crystal glasses 40, the control apparatus 10 senses (acquires information on the opening and closing timings) the opening and closing timings of the right and left eye shutters on the liquid crystal glasses 40. The configuration (see FIG. 1) of the surgery assistance system 1 and the timing chart (FIG. 11) of the fifth embodiment are similar to those of the fourth embodiment.

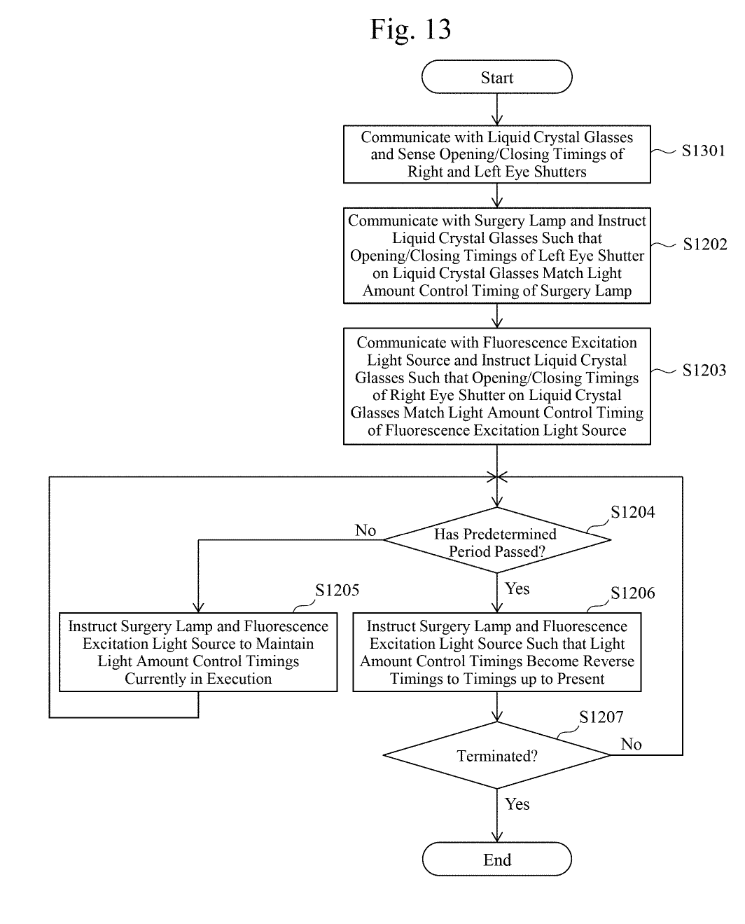

FIG. 13 is a flowchart describing process contents (contents of the illumination control program 1061) of the control apparatus 10 according to the fifth embodiment. FIG. 13 differs from FIG. 12 only in Step 1301, and the other processes of FIG. 13 are similar to those of FIG. 12.

### (i) Step 1301

For example, the wearing target, such as the doctor (the operator) who wears the liquid crystal glasses 40 and is going to perform a surgery, turns ON power of the liquid crystal glasses 40, turns ON power of the control apparatus 10, and sets a mode to a surgery mode. Then, the liquid crystal glasses 40 start the opening and closing behaviors of the right and left eye shutters at the preliminarily set timing (for example, every 1/60 seconds). The controller 101 in the control apparatus 10 communicates with the liquid crystal glasses 40 and senses the opening and closing timings of the left eye shutter and the right eye shutter on the liquid crystal glasses 40. For example, a memory (not illustrated) in the liquid crystal glasses 40 holds the information on the opening and closing timings of the liquid crystal shutters of the right and left eyes and a controller (a processor, not illustrated) acquires the information on the opening and closing timings of the liquid crystal shutters from this memory, thus actually opening and closing the liquid crystal shutters of the right and left eyes. Therefore, the control apparatus 10 communicates with the controller in the liquid crystal glasses 40 using the communication apparatus 105 to acquire the information on the opening and closing timings of the currently behaving liquid crystal shutters. The opening and closing timings of the shutters of the right and left eyes thus acquired are as illustrated in FIG. 11 (for example, opened/closed at every 1/60 seconds in alternation). While FIG. 11 illustrates the case where the light amount control timing of the surgery lamp 20 and the light amount control timing of the fluorescence excitation light source 30 occur in alternation as one example, the light amount control timing of the surgery lamp 20 may partially overlap with the light amount control timing of the fluorescence excitation light source 30.

### (ii) Steps 1202 to 1207

Since being similar to those of FIG. 12, the process contents of Steps 1202 to 1207 are omitted. While the processes of Steps 1201 to 1203 may be performed simultaneously in the fourth embodiment, the process of Step 1301 needs to be performed first to acquire the information on the opening and closing timings of the right and left eye shutters on the liquid crystal glasses 40 in the fifth embodiment. The light amount control timings of the surgery lamp 20 and the fluorescence excitation light source 30 are controlled according to the information. Note that the processes of Steps 1202 and 1203 may be performed simultaneously.

In the fifth embodiment, the eyesight restricting apparatus (for example, the liquid crystal glasses 40) performs the open/close control on the eyesight of the left eye and the eyesight of the right eye of, for example, the person (for example, the doctor and the operator) who wears the eyesight restricting apparatus in alternation at every predetermined time interval. The eyesight of the left eye can also be referred to as the first eyesight, the eyesight of the right eye as the second eyesight, or conversely, the eyesight of the right eye can also be referred to as the first eyesight, and the eyesight of the left eye as the second eyesight. While the control apparatus 10 controls this open/close control in the fourth embodiment, the eyesight restricting apparatus performs this open/close control by itself in the fifth embodiment. Meanwhile, the control apparatus 10 controls the irradiation timings of the first light at the first light amount and the second light amount and the irradiation timings of the second light at the third light amount and the fourth light amount by the light sources (the surgery lamp 20 and the fluorescence excitation light source 30). For example, the timing of light amount control of the first light and the timing of light amount control of the second light are different from one another. For example, the timing of light amount control of the first light and the timing of light amount control of the second light may occur in alternation or may partially overlap with one another. In such fifth embodiment, the control apparatus 10 communicates (for example, the wireless communications) with the eyesight restricting apparatus (for example, the liquid crystal glasses 40) to acquire the information on the opening and closing timings of the first eyesight and the second eyesight of the eyesight restricting apparatus (the liquid crystal glasses 40). Accordingly, the control apparatus 10 can acquire the information on the opening and closing timings of the respective eyesights of the eyesight restricting apparatus (the liquid crystal glasses 40). The control apparatus 10 controls the light amounts of the irradiated lights by the light sources (the surgery lamp 20 and the fluorescence excitation light source 30) such that the first light is irradiated at the first light amount at a timing (for example, timings in alternation) different from the irradiation of the second light at the third light amount (For example, when the irradiation of the first light at the first light amount and the irradiation of the second light at the third light amount are performed at alternate timings, the irradiation timing of the first light at the second light amount and the irradiation timing of the second light at the third light amount become identical.); the opening and closing timings of the first eyesight match the timing of light amount control of the irradiated first light; and the opening and closing timings of the second eyesight match the timing of light amount control of the irradiated second light. According to the fifth embodiment, the eyesight restricting apparatus (the liquid crystal glasses 40) needs not to control the behaviors according to the control signal from the control apparatus 10. This allows further simplification of the apparatus internal configuration of the eyesight restricting apparatus (the liquid crystal glasses 40). According to the fifth embodiment, similar to the fourth embodiment, while the excitation light is irradiated at the fourth light amount (the light amount is smaller than the third light amount), the biological tissue is irradiated with the visible light at the first light amount and while the excitation light is irradiated at the third light amount, the biological tissue is irradiated with the visible light at the second light amount (The light amount is smaller than the first light amount). This reduces the difference between bright and dark between the surgery lamp and the fluorescence, thereby ensuring easily seeing both the operative field and the fluorescence.

While the eyesight restricting apparatus (for example, the liquid crystal glasses 40) may perform the open/close control on the eyesight of the left eye and the eyesight of the right eye in alternation as described above, the open/close control may be performed such that the opening and closing timings of the eyesight of the left eye and the eyesight of the right eye at this eyesight restricting apparatus partially overlap with one another. The case where the timings partially overlap with one another can include, for example, the case where there are time slots in which both of the eyesight of the right eye and the eyesight of the left eye are in the open state and the closed state, or there is a time slot in which both eyesights are in the closed state or the open state.

### (6) Sixth Embodiment

The sixth embodiment relates to an operating microscope to which, for example, the principles of the fourth and fifth embodiments are applied. To perform the surgery on the patient (for example, a human and an animal) using this operating microscope, for example, a fluorescer (as one example, 5-aminolevulinic acid (5-ALA)) reacting to excitation light is administrated to the patient at a predetermined time before the surgery. For example, a local administration by oral administration and injection is considered as the administration method. Thus, this operating microscope is used after administrating, for example, the predetermined fluorescer.

<Appearance Configuration>

An example of an appearance of the operating microscope system and an example of an appearance of the operating microscope according to the sixth embodiment are identical to those of the third embodiment, which are each illustrated in FIG. 7 and FIG. 8. In view of this, the following omits descriptions other than descriptions of the operating microscope light source 80 and the fluorescence excitation light source 30 described below.

Similar to the fourth embodiment, while a white LED light source with an illuminance of, for example, 60000 lux, brightness sufficient for surgery, is usable as the operating microscope light source 80, the illumination may have the illuminance of at least, for example, 500 to 1000 lux. The operating microscope light source 80 is coupled to the control apparatus 10 such that the control apparatus 10 controls the timings (a first illumination period at the first light amount with the large light amount and a second illumination period at the second light amount during which the light amount is smaller than that of the first illumination period) of the light amount control (the light amount adjustment). For example, the operating microscope light source 80, which includes a driver (not illustrated), controls a current value flowing through the LED in accordance with the light amount control timing instructed by the control apparatus 10 to behave so as to repeat the increase and decrease of the light amount at every predetermined period (for example, 1/60 seconds).

Similar to the fourth embodiment, the fluorescence excitation light source 30 is a light source, for example, that emits light having a wavelength within a predetermined bandwidth. For example, with the use of the 5-ALA as the fluorescer, as the fluorescence excitation light source 30, a light source with a narrow bandwidth emitting a light (an ultraviolet light) with a wavelength of, for example, 385 nm to 425 nm is usable. Similar to the operating microscope light source 80, the fluorescence excitation light source 30 is coupled to the control apparatus 10 such that the control apparatus 10 controls the timings (a third illumination period at the third light amount with the large light amount and a fourth illumination period at the fourth light amount during which the light amount is smaller than that of the third illumination period) of the light amount control (the light amount adjustment). For example, the fluorescence excitation light source 30, which includes a driver and a controller (not illustrated), controls a current value flowing through the fluorescence excitation light source (for example, the LED) in accordance with the light amount control timing instructed by the control apparatus 10 to behave so as to repeat the increase and decrease of the light amount at every predetermined period (for example, 1/60 seconds). For example, the light-on and off timing of the surgery lamp 20 at the first light amount and the light-on and off timing of the fluorescence excitation light source 30 at the third light amount are controlled so as to occur in alternation at every predetermined period (for example, 1/60 seconds). The light-on and off timings of the surgery lamp 20 at the second light amount and the light-on and off timings of the fluorescence excitation light source 30 at the fourth light amount are controlled so as to occur in alternation at every predetermined period (for example, 1/60 seconds).

As one example, the control apparatus 10 transmits the control signal to the operating microscope light source 80, the fluorescence excitation light source 30, and the driver (not illustrated) and the controller (not illustrated) of the liquid crystal shutters 86 to instruct these apparatuses on the light amount control timings of these light sources and the opening and closing timings of the liquid crystal shutters 86. For example, when the above-described first eyesight is in the light shielding state (the left eye shutter closed) and the second eyesight is in the light transmission state (the right eye shutter opened) at the liquid crystal shutters 86, the control apparatus 10 controls the respective behaviors such that the operating microscope light source 80 lights on at the second light amount (The light amount is smaller than the first light amount.) and the fluorescence excitation light source 30 lights on at the third light amount (The light amount is larger than the fourth light amount). Additionally, for example, when the above-described first eyesight is in the light transmission state (the left eye shutter opened) and the second eyesight is in the light shielding state (the right eye shutter closed), the control apparatus 10 controls the respective behaviors such that the operating microscope light source 80 lights on at the first light amount (The light amount is larger than the second light amount.) and the fluorescence excitation light source 30 lights on at the fourth light amount (The light amount is smaller than the third light amount).

<Optical System Configuration>

A configuration of an optical system of the operating microscope according to the sixth embodiment can employ the configuration example (see FIG. 9) of the optical system of the operating microscope 70 according to the third embodiment.

The light amount control of the illuminating light from the operating microscope light source 80, the light amount control of the excitation light from the fluorescence excitation light source 30, and the opening and closing of the right and left eye shutters on the liquid crystal shutters 86 are controlled at the timings illustrated in FIG. 11. While FIG. 11 illustrates the case where the light amount control timing of the operating microscope light source 80 and the light amount control timing of the fluorescence excitation light source 30 occur in alternation as one example, the light amount control timing of the surgery lamp 20 may partially overlap with the light amount control timing of the fluorescence excitation light source 30.

For example, the operating microscope light source 80 is lit on (at this time, the fluorescence excitation light source is lit on at the fourth light amount (dark)) at the first light amount (for example, the maximum light amount) for a predetermined period (for example, 1/60 seconds), and the tissue BT is irradiated with the illuminating light at the first light amount (bright) emitted from the operating microscope light source 80 via the illuminating optical system 21 and the mirror 22. The reflected light from the tissue BT, for example, becomes the two lights corresponding to the right and left eyes observed at the eyepieces 71, and the two lights are introduced to the liquid crystal shutters 86 via the objective optical systems 81, the first prism 82, the zoom optical system 83, the second prism 84, and the third prism 85. In the case where the operating microscope light source 80 lights on at this first light amount, the liquid crystal shutters 86 are, for example, controlled such that the first eyesight (for example, the eyesight at the left eye shutter) enters the light transmission state and the second eyesight (for example, the eyesight at the right eye shutter) enters the light shielding state. Therefore, the reflected light from the tissue BT passes through only the first eyesight restrictor (the left eye shutter) and reaches only the one eyepiece region (for example, the first eyepiece including the left eye ocular lens) of the eyepieces 71 via, for example, the notch filter F.

When the light-on of the operating microscope light source 80 at the first light amount passes for the predetermined period (for example, the above-described 1/60 seconds), the control is performed such that the operating microscope light source 80 lights on at the second light amount smaller than the first light amount and the fluorescence excitation light source 30 lights on at the third light amount (bright) for the predetermined period (for example, 1/60 seconds). Additionally, the control is performed such that the first eyesight restrictor (the left eye shutter) of the liquid crystal shutters 86 enters the light shielding state and the second eyesight restrictor (the right eye shutter) enters the light transmission state. During this period, the tissue BT is irradiated with the excitation light at the third light amount (bright) from the fluorescence excitation light source 30 via, for example, the excitation optical system 31 and the mirror 32. The tissue BT is also irradiated with the illuminating light (the second light amount) from the operating microscope light source 80 via the illuminating optical system 21 and the mirror 22. The irradiation of the tissue BT with the excitation light 33 at the third light amount (bright) emits the fluorescence from the affected part (for example, the tumor part) of the tissue BT. This fluorescence from the affected part, for example, becomes two lights corresponding to the right and left eyes observed at the eyepieces 71, and the two lights are introduced to the liquid crystal shutters 86 via the objective optical systems 81, the first prism 82, the zoom optical system 83, the second prism 84, and the third prism 85. The light (the reflected light), which is the illuminating light from the operating microscope light source 80 reflected by the tissue BT, also becomes two lights corresponding to the right and left eyes observed at the eyepieces 71, and the two lights are introduced to the liquid crystal shutters 86 via the objective optical systems 81, the first prism 82, the zoom optical system 83, the second prism 84, and the third prism 85.

In the case where the fluorescence excitation light source 30 lights on at the third light amount (bright), as described above, since the liquid crystal shutters 86 are controlled such that the first eyesight restrictor (the left eye shutter) enters the light shielding state and the second eyesight restrictor (the right eye shutter) enters the light transmission state, the fluorescence and the reflected light pass through only the second eyesight restrictor (the right eye shutter). Further, since the reflected light of the excitation light via the tissue BT is removed by, for example, the notch filter F, only the fluorescence and the reflected light of the illuminating light (the second light amount) reflected by the tissue BT reach the other eyepiece region (for example, the second eyepiece including the right eye ocular lens) of the eyepieces 71.

The above-described processes are switchingly repeated at every predetermined period (for example, 1/60 seconds). When the operating microscope light source 80 lights on (the fluorescence excitation light source 30 lights on at the fourth light amount (dark)) at the first light amount and the left eye shutter is in the light transmission state (The right eye shutter is in the light shielding state.), the doctor or a similar person (the operator) who looks through the eyepieces 71 can brightly observe the tissue BT by the left eye. Meanwhile, when the fluorescence excitation light source 30 lights on (the surgery lamp 20 lights on at the second light amount) at the third light amount (bright) and the right eye shutter is in the light transmission state (The left eye shutter is in the light shielding state.), the doctor or a similar person (the operator) who looks through the eyepieces 71 can observe the tumor part (the affected part) emitting the fluorescence in the relatively dark eyesight by the right eye. By switchingly controlling these states at every constant period (for example, every 1/60 seconds), the image of the right eye and the image of the left eye are seen to be combined in a pseudo manner in the brain of the doctor or a similar person (the operator), and thus the fluorescence from the tumor part (the affected part) of the tissue BT is seen to be highlighted (The fluorescence from the tissue BT is not negated by the operating microscope light source 80 but can be clearly seen). The operating microscope light source 80 is not lit off but just the light amount is lowered while the fluorescence excitation light source 30 lights on at the third light amount (bright). This reduces the difference between bright and dark between an operating microscope illumination and the fluorescence, thereby ensuring easily seeing both the operative field and the fluorescence.

While this embodiment synchronizes the operating microscope light source 80 with the left eye shutter on the liquid crystal shutters 86 and synchronizes the fluorescence excitation light source 30 with the right eye shutter on the liquid crystal shutters 86, the synchronization timing may be reversed. For example, while the operating microscope light source 80 according to this embodiment uses the light source that can perform coaxial illumination on the tissue BT, a light source that can perform a ring illumination on the tissue BT may be used. For example, with the use of the ring illumination, the operating microscope light source 80 according to this embodiment can evenly irradiate the tissue BT from all directions.

<Details of Synchronization Timing Control and Process Contents>

The controls of the light amount control timing of the operating microscope light source 80, the light amount control timing of the fluorescence excitation light source 30, and the opening and closing timings of the liquid crystal shutters of the sixth embodiment are, for example, as shown by the timing chart of FIG. 11 and its description; therefore, the detailed description is omitted here. Note that in FIG. 11 and its description, “the surgery lamp” can be read as “the operating microscope light source” and “the liquid crystal glasses” as “the liquid crystal shutters.”

The control process contents by the control apparatus 10 in the sixth embodiment are, for example, as illustrated in the flowchart of FIG. 12 and its description; therefore, the detailed description is omitted here. Note that in FIG. 12 and its description, “the surgery lamp” can be read as “the operating microscope light source” and “the liquid crystal glasses” as “the liquid crystal shutters.” Moreover, while the fourth embodiment is described that the control apparatus 10 and the liquid crystal glasses 40 perform the wireless communications, in the sixth embodiment, for example, the control apparatus 10 and the liquid crystal shutters 86 may be coupled by wires for ensuring communications (Note that this does not deny a possibility of wireless communications).

The sixth embodiment is an application of the principle according to the fourth embodiment to the operating microscope. While this embodiment refers to the microscope as “the operating microscope,” the operating microscope is not limited to the surgery application and is widely applicable to the general medical practice. The operating microscope 70 includes a light introduction mechanism (for example, the illumination light path L and the excitation light path E), the eyepieces 71, a usual light path (the observation light path A), the light dividers (the objective optical systems 81), the eyesight restrictors (for example, the liquid crystal shutters 86), and the control apparatus 10. The light introduction mechanism introduces the first light and the second light emitted from the light sources (for example, the operating microscope light source 80 and the fluorescence excitation light source 30) to the biological tissue. The usual light path guides the light from the biological tissue to the eyepieces 71. The light dividers, which are disposed on the usual light path, divide the light from the biological tissue into lights of the right and left eyesights of the user. The eyesight restrictors are disposed between the light dividers and the eyepieces. The control apparatus 10 controls the behaviors of the light sources and the behaviors of the eyesight restrictors. The control apparatus 10 performs the open/close control on the first eyesight and the second eyesight (the eyesight of the left eye can be defined as the first eyesight and the eyesight of the right eye as the second eyesight, or conversely the eyesight of the right eye can be defined as the first eyesight and the eyesight of the left eye as the second eyesight) of the eyesight restrictor in alternation at every predetermined time interval. Then, the control apparatus 10 differentiates (For example, the timings are configured to occur in alternation. At this time, the first light at the second light amount is introduced simultaneously with the introduction of the second light at the third light amount, and the first light at the first light amount is introduced simultaneously with the introduction of the second light at the fourth light amount.) the timing to introduce (irradiate) the first light at the first light amount from the timing to introduce (irradiate) the second light at the third light amount; matches the opening and closing timings of the first eyesight with the timings of the introduction of the first light at the first light amount and the introduction of the first light at the second light amount; and matches the opening and closing timings of the second eyesight with the timings of the introduction of the second light at the third light amount and the introduction of the second light at the fourth light amount. The operating microscope 70 according to this embodiment brightly irradiates the biological tissue, thereby ensuring the surgery while directly confirming the tumor part (the affected part). According to this embodiment, similar to the fourth embodiment, while the excitation light is irradiated at the third light amount, the biological tissue is irradiated with the visible light at the second light amount, and while the biological tissue is irradiated with the visible light at the first light amount, the biological tissue is irradiated with the excitation light at the fourth light amount. This reduces the difference between bright and dark between the surgery lamp and the fluorescence, thereby ensuring easily seeing both the operative field and the fluorescence. While this embodiment describes the example where the timing of light amount control of the introduced first light occurs “in alternation” with the timing of light amount control of the introduced second light, the timings need not to occur “in alternation” and both timings only need to be different. For example, the timings may occur at cycles in phases opposite to one another, or both timings may partially overlap with one another.

The control apparatus 10 communicates with the eyesight restrictors (for example, the liquid crystal shutters 86) to perform the open/close control on the eyesight of the left eye and the eyesight of the right eye. The opening and closing timings of the eyesight of the left eye and the eyesight of the right eye at these eyesight restrictors, for example, may be controlled so as to occur in alternation or may partially overlap with one another. The case where the timings partially overlap with one another can include, for example, the case where there are time slots in which both of the eyesight of the right eye and the eyesight of the left eye are in the open state and the closed state, or there is a time slot in which both eyesights are in the closed state or the open state.

### (7) Other Embodiments

For example, the respective embodiments may use not only the fluorescence but also phosphorescence as the second light, and the second light may be a light emitted from the tissue BT by the irradiation of the tissue BT with the light.

In the above-described respective embodiments, for example, the user can perform the surgery on the fluorescent part in the tissue of the organism (for example, an animal) with the surgery lamp and the operating microscope illumination lit on and also can confirm the anatomical structure of this organism other than this fluorescence part.

The functions of the respective embodiments can also be achieved by a program code of software. In this case, a storage medium recording the program codes is provided to the system or the apparatus, and a computer (or a CPU and an MPU) in the system or the apparatus reads the program codes stored in the storage medium. In this case, the program codes themselves read from the storage medium achieve the above-described functions of the embodiments, and this embodiment includes the program codes themselves and the storage medium storing the program codes. As the storage medium supplying such program codes, for example, a flexible disk, a CD-ROM, a DVD-ROM, a hard disk, an optical disk, a magneto-optical disk, a CD-R, a magnetic tape, a non-volatile memory card, and a ROM are used.

An operating system (OS) operating on the computer or a similar system may perform a part of or all of the actual processes based on instructions from the program codes, and the above-described functions of the embodiments may be achieved by the processes. Furthermore, after the program codes read from the storage medium are written to a memory in the computer, the CPU in the computer or a similar system may perform a part of or all of the actual processes based on the instructions from the program codes, and the above-described functions of the embodiments may be achieved by the processes.

Furthermore, the program codes of the software, which achieve the functions of the embodiments, may be distributed over a network, storage means such as a hard disk and a memory in the system or the apparatus or the storage medium such as the CD-RW and the CD-R may store the program codes, and the computer (or the CPU and the MPU) in the system or the apparatus may read and perform the program codes stored in this storage means and this storage medium for use.

The processes and techniques described here are not essentially related to any particular apparatus, and can be implemented by any suitable combination of components. Furthermore, general-purpose, various types of apparatuses can be used in accordance with the method described here. For executing steps of the method described here, it may be beneficial to construct a dedicated apparatus. Further, various inventions can be made by properly combining the plurality of constituents disclosed in the embodiments. For example, some constituents may be omitted from all of the constituents shown in the embodiments. Moreover, components in different embodiments may be suitably combined together.

Other implementations of the present invention will be made apparent for those having ordinary knowledge in the technical field from the examination of the specification and the embodiments of the present invention disclosed herein. The various configurations and/or components of the described embodiments can be used independently or in any combination.

### REFERENCE SIGNS LIST

1 Surgery assistance system
10 Control apparatus
20 Surgery lamp (light source for surgery)
30 Fluorescence excitation light source
40 Liquid crystal glasses
101 Controller
102 Input apparatus
103 Output apparatus
104 Storage
106 Memory
1061 Illumination control program
60 Operating microscope system
61 Coupling arm
62 Support arm
63 Distal end member
64 Link mechanism
70 Operating microscope
74 Eyepiece
72 Light flux receiver
73 Chassis
80 Operating microscope light source
81 Objective optical system
86 Liquid crystal shutter

1. A light irradiation system comprising:
a light source configured to emit a first light and a second light with which a biological tissue is irradiated;
an eyesight restricting apparatus that includes a first eyesight restrictor and a second eyesight restrictor configured to control eyesights of a target; and
a control apparatus configured to control a behavior of the light source and a behavior of the eyesight restricting apparatus,
wherein the control apparatus is configured to perform an open/close control on a first eyesight of the first eyesight restrictor and a second eyesight of the second eyesight restrictor at every predetermined time interval, the control apparatus being configured to control a light irradiation behavior by the light source such that:
opening and closing timings of any one of the first eyesight and the second eyesight match a timing of a light amount control of the irradiated first light; and
opening and closing timings of an eyesight other than the eyesight matched with the timing of the light amount control of the first light match a timing of a light amount control of the irradiated second light.

2. The light irradiation system according to claim 1,
wherein the light source includes an LED light source and an excitation light source, the LED light source being configured to emit a white light as the first light, the excitation light source being configured to emit an excitation light with a predetermined wavelength as the second light.

3. The light irradiation system according to claim 1,
wherein the first light is a visible light, and the second light is an excitation light.

4. The light irradiation system according to claim 2,
wherein the light source is configured to irradiate the biological tissue with the excitation light, a fluorescer having been administrated to the biological tissue.

5. The light irradiation system according to claim 2,
wherein the excitation light has a wavelength of 385 nm to 425 nm.

6. The light irradiation system according to claim 1,
wherein the eyesight restricting apparatus includes liquid crystal glasses configured to switch the first eyesight and the second eyesight between a light transmission and a light shielding.

7. The light irradiation system according to claim 1,
wherein the control apparatus is configured to perform wireless communications with the eyesight restricting apparatus to perform the open/close control on the first eyesight and the second eyesight in alternation.

8. The light irradiation system according to claim 1,
wherein the light amount control of the first light is an ON and OFF control of the first light irradiation,
the light amount control of the second light is an ON and OFF control of the second light irradiation, and
the control apparatus is configured to perform the open/close control on the first eyesight of the first eyesight restrictor and the second eyesight of the second eyesight restrictor at every predetermined time interval, the control apparatus being configured to control a light-on of the light and a light-off of the light by the light source such that:
the opening and closing timings of any one of the first eyesight and the second eyesight match the ON and OFF timings of the first light irradiation; and
the opening and closing timings of the eyesight other than the eyesight matched with the ON and OFF timings of the first light irradiation match the ON and OFF timings of the second light irradiation.

9. The light irradiation system according to claim 1,
wherein the control apparatus is configured to perform the open/close control on the first eyesight of the first eyesight restrictor and the second eyesight of the second eyesight restrictor at every predetermined time interval, the control apparatus being configured to control a light amount of the first light and a light amount of the second light by the light source such that:
the opening and closing timings of any one of the first eyesight and the second eyesight match a switching control timing of an irradiation of the first light at a first light amount and an irradiation of the first light at a second light amount, the second light amount being smaller than the first light amount; and
the opening and closing timings of the eyesight other than the eyesight matched with the switching control timing of the first light match a switching control timing of an irradiation of the second light at a third light amount and an irradiation of the second light at a fourth light amount, the fourth light amount being smaller than the third light amount.

10. A control apparatus that controls a behavior of a light source and a behavior of an eyesight restricting apparatus, the light source irradiating a biological tissue with a first light and a second light, the eyesight restricting apparatus including a first eyesight restrictor and a second eyesight restrictor to control eyesights of a target, the control apparatus comprising:
a memory that stores a program to control the behavior of the light source and the behavior of the eyesight restricting apparatus; and
a controller configured to read the program from the memory to execute the program,
wherein the controller is configured to perform an open/close control on a first eyesight of the first eyesight restrictor and a second eyesight of the second eyesight restrictor at every predetermined time interval, the controller being configured to control a light irradiation behavior of the light source such that:
opening and closing timings of any one of the first eyesight and the second eyesight match a timing of a light amount control of the irradiated first light; and
opening and closing timings of an eyesight other than the eyesight matched with the timing of the light amount control of the first light match a timing of a light amount control of the irradiated second light.

11. The control apparatus according to claim 10, comprising
a communication apparatus configured to perform wireless communications with the eyesight restricting apparatus,
wherein the controller is configured to perform the wireless communications with the eyesight restricting apparatus via the communication apparatus to perform the open/close control on the first eyesight and the second eyesight.

12. The control apparatus according to claim 10,
wherein the light amount control of the first light is an ON and OFF control of the first light irradiation,
the light amount control of the second light is an ON and OFF control of the second light irradiation, and
the controller is configured to perform the open/close control on the first eyesight of the first eyesight restrictor and the second eyesight of the second eyesight restrictor at every predetermined time interval, the controller being configured to control a light-on of the light and a light-off of the light by the light source such that:
the opening and closing timings of any one of the first eyesight and the second eyesight match the ON and OFF timing of the first light irradiation; and
the opening and closing timings of the eyesight other than the eyesight matched with the ON and OFF timing of the first light irradiation match the ON and OFF timing of the second light irradiation.

13. The control apparatus according to claim 10,
wherein the controller is configured to perform the open/close control on the first eyesight of the first eyesight restrictor and the second eyesight of the second eyesight restrictor at every predetermined time interval, the controller being configured to control a light amount of the first light and a light amount of the second light by the light source such that:
the opening and closing timings of any one of the first eyesight and the second eyesight match a switching control timing of an irradiation of the first light at a first light amount and an irradiation of the first light at a second light amount, the second light amount being smaller than the first light amount; and
the opening and closing timings of the eyesight other than the eyesight matched with the switching control timing of the first light match a switching control timing of an irradiation of the second light at a third light amount and an irradiation of the second light at a fourth light amount, the fourth light amount being smaller than the third light amount.

14. A light irradiation system comprising:
a light source configured to irradiate a biological tissue with a first light and a second light;
an eyesight restricting apparatus that includes a first eyesight restrictor and a second eyesight restrictor configured to control eyesights of a target; and
a control apparatus configured to control a behavior of the light source, wherein the eyesight restricting apparatus is configured to perform an open/close control on a first eyesight of the first eyesight restrictor and a second eyesight of the second eyesight restrictor at every predetermined time interval, and
the control apparatus is configured to communicate with the eyesight restricting apparatus to acquire information on opening and closing timings of the first eyesight and the second eyesight, the control apparatus being configured to control a light irradiation behavior by the light source such that:
opening and closing timings of any one of the first eyesight and the second eyesight match a timing of a light amount control of the irradiated first light; and
opening and closing timings of an eyesight other than the eyesight matched with the timing of the light amount control of the first light match a timing of a light amount control of the irradiated second light.

15. The light irradiation system according to claim 14,
wherein the control apparatus is configured to control the light irradiation behavior by the light source based on the information on opening and closing timings of the first eyesight or the second eyesight.

16. The light irradiation system according to claim 14,
wherein the control apparatus is configured to perform wireless communications with the eyesight restricting apparatus to acquire the information on the timings of the open/close control of the first eyesight and the second eyesight.

17. The light irradiation system according to claim 14,
wherein the light amount control of the first light is an ON and OFF control of the first light irradiation,
the light amount control of the second light is an ON and OFF control of the second light irradiation, and
the controller is configured to perform the open/close control on the first eyesight of the first eyesight restrictor and the second eyesight of the second eyesight restrictor at every predetermined time interval, the controller being configured to control a light-on of the light and a light-off of the light by the light source such that:
the opening and closing timings of any one of the first eyesight and the second eyesight match the ON and OFF timings of the first light irradiation; and
the opening and closing timings of the eyesight other than the eyesight matched with the ON and OFF timings of the first light irradiation match the ON and OFF timings of the second light irradiation.

18. The light irradiation system according to claim 14,
wherein the controller is configured to perform the open/close control on the first eyesight of the first eyesight restrictor and the second eyesight of the second eyesight restrictor at every predetermined time interval, the controller being configured to control a light amount of the first light and a light amount of the second light by the light source such that:
the opening and closing timings of any one of the first eyesight and the second eyesight match a switching control timing of an irradiation of the first light at a first light amount and an irradiation of the first light at a second light amount, the second light amount being smaller than the first light amount; and
the opening and closing timings of the eyesight other than the eyesight matched with the switching control timing of the first light match a switching control timing of an irradiation of the second light at a third light amount and an irradiation of the second light at a fourth light amount, the fourth light amount being smaller than the third light amount.

19. A control apparatus that controls a behavior of a light source, the light source irradiating a biological tissue with a first light and a second light, the control apparatus comprising:
a memory that stores a program to control the behavior of the light source; and
a controller configured to read the program from the memory to execute the program,
wherein the controller is configured to acquire information on a timing of an open/close control from an eyesight restricting apparatus, the eyesight restricting apparatus including a first eyesight restrictor and a second eyesight restrictor to control eyesights of a target, the open/close control being performed on a first eyesight of the first eyesight restrictor and a second eyesight of the second eyesight restrictor at every predetermined time interval, the controller being configured to control a light irradiation behavior by the light source such that:
opening and closing timings of any one of the first eyesight and the second eyesight match a timing of a light amount control of the irradiated first light; and
opening and closing timings of an eyesight other than the eyesight matched with the timing of the light amount control of the first light match a timing of a light amount control of the irradiated second light.

20. The control apparatus according to claim 19, comprising
a communication apparatus configured to perform wireless communications with the eyesight restricting apparatus,
wherein the controller is configured to perform the wireless communications with the eyesight restricting apparatuses via the communication apparatus to acquire the information on the timings of the open/close control on the first eyesight and the second eyesight.

21. The control apparatus according to claim 19,
wherein the light amount control of the first light is an ON and OFF control of the first light irradiation,
the light amount control of the second light is an ON and OFF control of the second light irradiation, and
the controller is configured to perform the open/close control on the first eyesight of the first eyesight restrictor and the second eyesight of the second eyesight restrictor at every predetermined time interval, the controller being configured to control a light-on of the light and a light-off of the light by the light source such that:
the opening and closing timings of any one of the first eyesight and the second eyesight match the ON and OFF timings of the first light irradiation; and
the opening and closing timings of the eyesight other than the eyesight matched with the ON and OFF timings of the first light irradiation match the ON and OFF timings of the second light irradiation.

22. The control apparatus according to claim 19,
wherein the controller is configured to perform the open/close control on the first eyesight of the first eyesight restrictor and the second eyesight of the second eyesight restrictor at every predetermined time interval, the controller being configured to control a light amount of the first light and a light amount of the second light by the light source such that:
the opening and closing timings of any one of the first eyesight and the second eyesight match a switching control timing of an irradiation of the first light at a first light amount and an irradiation of the first light at a second light amount, the second light amount being smaller than the first light amount; and
the opening and closing timings of the eyesight other than the eyesight matched with the switching control timing of the first light match a switching control timing of an irradiation of the second light at a third light amount and an irradiation of the second light at a fourth light amount, the fourth light amount being smaller than the third light amount.

23. A light irradiation control method that controls a behavior of a light source and a behavior of an eyesight restricting apparatus by a control apparatus, the light source emitting a first light and a second light, the eyesight restricting apparatus including a first eyesight restrictor and a second eyesight restrictor to control eyesights of a target, the light irradiation control method comprising:
performing an open/close control on a first eyesight of the first eyesight restrictor and a second eyesight of the second eyesight restrictor at every predetermined time interval by the control apparatus; and
controlling a light irradiation behavior by the light source by the control apparatus such that:
opening and closing timings of any one of the first eyesight and the second eyesight match a timing of a light amount control of the irradiated first light; and
opening and closing timings of an eyesight other than the eyesight matched with the timing of the light amount control of the first light match a timing of a light amount control of the irradiated second light.

24. The light irradiation control method according to claim 23,
wherein the light amount control of the first light is an ON and OFF control of the first light irradiation,
the light amount control of the second light is an ON and OFF control of the second light irradiation, and
in the controlling the light irradiation behavior by the light source, the control apparatus is configured to perform the open/close control on the first eyesight of the first eyesight restrictor and the second eyesight of the second eyesight restrictor at every predetermined time interval, the control apparatus being configured to control a light-on of the light and a light-off of the light by the light source such that:
the opening and closing timings of any one of the first eyesight and the second eyesight match the ON and OFF timings of the first light irradiation; and
the opening and closing timings of the eyesight other than the eyesight matched with the ON and OFF timings of the first light irradiation match the ON and OFF timings of the second light irradiation.

25. The light irradiation control method according to claim 23,
wherein in the controlling the light irradiation behavior by the light source, the control apparatus is configured to perform the open/close control on the first eyesight of the first eyesight restrictor and the second eyesight of the second eyesight restrictor at every predetermined time interval, the control apparatus being configured to control a light amount of the first light and a light amount of the second light by the light source such that:
the opening and closing timings of any one of the first eyesight and the second eyesight match a switching control timing of an irradiation of the first light at a first light amount and an irradiation of the first light at a second light amount, the second light amount being smaller than the first light amount; and
the opening and closing timings of the eyesight other than the eyesight matched with the switching control timing of the first light match a switching control timing of an irradiation of the second light at a third light amount and an irradiation of the second light at a fourth light amount, the fourth light amount being smaller than the third light amount.

26. A light irradiation control method that controls a behavior of a light source emitting a first light and a second light by a control apparatus, the light irradiation control method comprising:
communicating with an eyesight restricting apparatus to acquire information on a timing of an open and close control from the eyesight restricting apparatus by the control apparatus, the eyesight restricting apparatus including a first eyesight restrictor and a second eyesight restrictor to control eyesights of a target, the open/close control being performed on a first eyesight of the first eyesight restrictor and a second eyesight of second eyesight restrictor at every predetermined time interval; and
controlling a light irradiation behavior by the light source by the control apparatus such that:
opening and closing timings of any one of the first eyesight and the second eyesight match a timing of a light amount control of the irradiated first light; and
opening and closing timings of an eyesight other than the eyesight matched with the timing of the light amount control of the first light match a timing of a light amount control of the irradiated second light.

27. The light irradiation control method according to claim 26,
wherein the light amount control of the first light is an ON and OFF control of the first light irradiation,
the light amount control of the second light is an ON and OFF control of the second light irradiation, and
in the controlling the light irradiation behavior by the light source, the control apparatus is configured to perform the open and close control on the first eyesight of the first eyesight restrictor and the second eyesight of the second eyesight restrictor at every predetermined time interval, the control apparatus being configured to control a light-on of the light and a light-off of the light by the light source such that:
the opening and closing timings of any one of the first eyesight and the second eyesight match the ON and OFF timings of the first light irradiation; and
the opening and closing timings of the eyesight other than the eyesight matched with the ON and OFF timings of the first light irradiation match the ON and OFF timings of the second light irradiation.

28. The light irradiation control method according to claim 26,
wherein in the controlling the light irradiation behavior by the light source, the control apparatus is configured to perform the open/close control on the first eyesight of the first eyesight restrictor and the second eyesight of the second eyesight restrictor at every predetermined time interval, the control apparatus being configured to control a light amount of the first light and a light amount of the second light by the light source such that:
the opening and closing timings of any one of the first eyesight and the second eyesight match a switching control timing of an irradiation of the first light at a first light amount and an irradiation of the first light at a second light amount, the second light amount being smaller than the first light amount; and
the opening and closing timings of the eyesight other than the eyesight matched with the switching control timing of the first light match a switching control timing of an irradiation of the second light at a third light amount and an irradiation of the second light at a fourth light amount, the fourth light amount being smaller than the third light amount.

29. An operating microscope apparatus comprising:
a light introduction mechanism configured to introduce a first light and a second light emitted from a light source to a biological tissue;
an eyepiece;
a usual light path configured to guide a light from the biological tissue to the eyepiece;
a light divider disposed on the usual light path, the light divider being configured to divide the light from the biological tissue into two lights;
a first light path and a second light path disposed between the light divider and the eyepiece, one light among the two lights passing through the first light path, another light among the two lights passing through the second light path;
an eyesight restrictor that includes a first eyesight restrictor disposed at the first light path and a second eyesight restrictor disposed at the second light path; and
a controller configured to control a behavior of the light source and a behavior of the eyesight restrictor,
wherein the controller is configured to perform an open/close control on a first eyesight of the first eyesight restrictor and a second eyesight of the second eyesight restrictor at every predetermined time interval, the controller being configured to match opening and closing timings of any one of the first eyesight and the second eyesight with a timing of a light amount control of the introduced first light, the controller being configured to match opening and closing timings of an eyesight other than the eyesight matched with the timing of the light amount control of the first light with a timing of a light amount control of the introduced second light.

30. The operating microscope apparatus according to claim 29,
wherein the light amount control of the first light is an ON and OFF control of the introduction of the first light,
the light amount control of the second light is an ON and OFF control of the introduction of the second light, and
the controller is configured to control the introduction of the light and a non-introduction of the light by the light source such that:
the first light irradiation and the second light irradiation are performed in alternation;
the opening and closing timings of any one of the first eyesight and the second eyesight match ON and OFF timings of the introduction of the first light; and
opening and closing timings of an eyesight other than the eyesight matched with the ON and OFF timings of the introduction of the first light match ON and OFF timings of the introduction of the second light.

31. The operating microscope apparatus according to claim 29,
wherein the controller is configured to perform the open/close control on the first eyesight of the first eyesight restrictor and the second eyesight of the second eyesight restrictor at every predetermined time interval, the controller being configured to match opening and closing timings of any one of the first eyesight and the second eyesight with a switching control timing of the introduction of the first light at a first light amount and the introduction of the first light at a second light amount, the second light amount being smaller than the first light amount, the controller being configured to match opening and closing timings of an eyesight other than the eyesight matched with the switching control timing of the first light with a switching control timing of the introduction of the second light at a third light amount and the introduction of the second light at a fourth light amount, the fourth light amount being smaller than the third light amount.

32. The operating microscope apparatus according to claim 29,
wherein the light source includes an LED light source and an excitation light source, the LED light source being configured to emit a white light as the first light, the excitation light source being configured to emit an excitation light with a predetermined wavelength as the second light.

33. The operating microscope apparatus according to claim 29,
wherein the first light is a visible light, and the second light is the excitation light.

34. The operating microscope apparatus according to claim 32,
wherein the light source is configured to irradiate the biological tissue with the excitation light, a fluorescer having been administrated to the biological tissue.

35. The operating microscope apparatus according to claim 32,
wherein the excitation light has a wavelength of 385 nm to 425 nm.

36. The operating microscope apparatus according to claim 29,
wherein the eyesight restrictor is a liquid crystal shutter configured to switch the first eyesight and the second eyesight between a light transmission and a light shielding.

37. The operating microscope apparatus according to claim 29,
wherein the light introduction mechanism includes a first light introduction light path and a second light introduction light path, the first light introduction light path being configured to irradiate the biological tissue with the first light, the second light introduction light path being configured to irradiate the biological tissue with the second light.

38. An illumination control system comprising:
a control apparatus configured to control a behavior of a light source; and
an eyesight restricting apparatus configured to control eyesights where two lights at different wavelengths transmit,
wherein the eyesight restricting apparatus is configured to adjust light amounts of the two lights transmitting the eyesights.

39. The illumination control system according to claim 38,
wherein the control apparatus is configured to control a light irradiation timing by the light source to an observation target, and
the eyesight restricting apparatus is configured to synchronize with the light irradiation timing to adjust the light amounts of the two lights transmitting the eyesights.

40. The illumination control system according to claim 38,
wherein the control apparatus is configured to control the light irradiation timing to an observation target by the light source based on timings of the light amount adjustment of the two lights by the eyesight restricting apparatus.

41. The illumination control system according to claim 38 wherein the eyesight restricting apparatus includes a first eyesight restrictor and a second eyesight restrictor, the first eyesight restrictor being configured to adjust a light amount of a light transmitting a first eyesight among the two lights, the second eyesight restrictor being configured to adjust a light amount of a light transmitting a second eyesight among the two lights.
